(19) United States
(12) Patent Application Publication
(10) Pub. No.: US 2024/0384232 A1
(43) Pub. Date: Nov. 21, 2024

(54) CLINICAL-GRADE THERAPEUTIC PROGENITORS GENERATED FROM TANKYRASE/PARP-INHIBITED PLURIPOTENT STEM CELL BANKS

(71) Applicant: THE JOHNS HOPKINS UNIVERSITY, Baltimore, MD (US)

(72) Inventors: Elias Zambidis, Baltimore, MD (US); Ludovic Zimmerlin, Baltimore, MD (US)

(73) Assignee: THE JOHNS HOPKINS UNIVERSITY, Baltimore, MD (US)

(21) Appl. No.: 18/785,343

(22) Filed: Jul. 26, 2024

Related U.S. Application Data
(63) Continuation of application No. PCT/US2023/011688, filed on Jan. 27, 2023.
(60) Provisional application No. 63/303,611, filed on Jan. 27, 2022.

(51) Int. Cl. C12N 5/0789

(52) U.S. Cl. CPC C12N 5/0647; C12N 2500/98; C12N 2501/235; C12N 2501/724; C12N 2501/727; C12N 2533/50

(57) ABSTRACT

The present disclosure relates to methods and compositions for generating both patient-specific and universal donor banked HLA-defined therapeutic progenitors in clinical grade, current good manufacturing practice (cGMP)-compliant conditions from a new class of tankyrase inhibitor-regulated naive human induced pluripotent stem cells (TIRN-hiPSCs).

### CROSS-REFERENCE TO RELATED APPLICATIONS

This application is a continuation of International Patent Application No. PCT/US2023/011688, filed Jan. 27, 2023, which claims priority to U.S. Provisional Patent Application Ser. No. 63/303,611 filed Jan. 27, 2022, the content of which are incorporated by reference in their entireties, and to which priority is claimed.

### STATEMENT REGARDING FEDERALLY SPONSORED RESEARCH

This invention was made with government support under grant HD082098awarded by the National Institutes of Health. The government has certain rights in the invention.

### 1. FIELD OF INVENTION

The present disclosure relates to methods for generating both patient-specific and universal donor banked HLA-defined therapeutic progenitors in clinical grade, current good manufacturing practice (cGMP)-compliant conditions where both the patient-specific and the universal donor banked HLA-defined therapeutic progenitors are derived from a new class of tankyrase/PARP inhibitor-regulated naïve human induced pluripotent stem cells (TIRN-hiPSCs).

### 2. BACKGROUND

Although human pluripotent stem cell (hPSC)-derived progenitors have wide impact for regenerative medicine, their broad clinical application via patient-specific approaches faces important challenges and limitations. For example, the current paradigm of autologous human induced pluripotent stem cell (hiPSC) therapeutics poses logistical and financial challenges. Specifically, the labor involved in screening individual hiPSC lines for high-quality clones and the costs associated with such screens makes patient-specific therapies inaccessible in health care systems with limited resources. Moreover, the costs of validating genomic integrity and functionality for individualized autologous hiPSC is not sustainable in the long term in a cost-conscious health care system. Even if such labor and cost challenges could be addressed, personalized patient-specific hiPSC lines are not readily available for numerous acute disorders requiring immediate therapeutic intervention (e.g., myocardial infarction, cerebrovascular stroke).

As an alternative to patient-specific cellular therapies, global efforts have begun to develop HLA-defined iPSC banks, including from inventories of clinical-grade, HLA-typed cord blood (CB). Clinical bone marrow transplantation (BMT) provides important paradigms to facilitate such hiPSC bank therapies. For example, the existing infrastructure of BMT routinely leverages partially HLA-matched, or haplo-identical HLA-matched hematopoietic stem cells.

Computational models have predicted that a small number of 80-200 HLA-defined hiPSC lines derived from existing HLA-typed cord or peripheral blood banks could generate allogeneic matches to serve the transplantation needs of the majorities of the populations in Japan, UK, and the USA. To prioritize immune-compatibility, HLA A, B, and DR loci (and O-negative blood group)-genotyped individuals from 2-10homozygous HLA haplotype donors could serve the needs of ˜5-30% of the population. Such ‘haplo’ banks of clinical-grade hiPSC lines could significantly expand therapeutic feasibility and reduce graft rejection, but are still likely to generate immune responses to hiPSC-derived tissues in non-autologous recipients. Moreover, the broad genetic diversity of the USA may limit the feasibility of HLA-matched hiPSC, even with the establishment of haplo banks.

In view of the foregoing, there remains a need in the field for cost effective methods for generating both patient-specific and universal donor banked HLA-defined therapeutic progenitors, particularly with respect to methods involving clinical grade, current good manufacturing practice (cGMP)-compliant conditions.

### 3. SUMMARY

In certain embodiments, the presently disclosed subject matter provides methods for generating a population of therapeutic progenitor cells under clinical grade, current good manufacturing practice (cGMP)-compliant conditions, comprising contacting a population of human induced pluripotent stem cells (hiPSCs) with a tankyrase/PARP-inhibitor-containing composition comprising (i) leukemia inhibitory factor (LIF), (ii) a Glycogen Synthase Kinase 3-β (GSK3β) signaling pathway inhibitor, (iii) a mitogen-activated protein kinase (MEK) signaling pathway inhibitor, (iv) a tankyrase/PARP inhibitor, and (v) a protein kinase C (PKC) inhibitor; the method further comprises performing feeder-free (FF) and xeno-free (XF) culture of the hiPSCs to generate a population of these therapeutic progenitor cells under clinical grade, cGMP-compliant conditions.

In certain embodiments, the tankyrase/PARP inhibitor is selected from: XAV939, IWR-1, G007-LK, JW55, AZ1366, JW 74, NVP-TNKS656 and combinations thereof. In certain embodiments, the GSK3β signaling pathway inhibitor is selected from: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M), N-(4-methoxybenzyl)-N′-(5-nitro-1,3-thiazol-2-yl)urea (AR-A014418) and combinations thereof. In certain embodiments, the MEK signaling pathway inhibitor is selected from: PD032590, CI-1040 (PD184352), cobimetinib (GDC-0973, XL518), Selumctinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 and combinations thereof. In certain embodiments, the PKC inhibitor is Go6983.

In certain embodiments, the hiPSCs are derived from primed hiPSCs. In certain embodiments, the priming of hiPSCs comprises contacting the hiPSCs with a serum replacer-containing media. In certain embodiments, priming the hiPSCs comprises contacting the hiPSCs with a ROCK inhibitor. In certain embodiments, the ROCK inhibitor is Y-27632.

In certain embodiments, the population of therapeutic progenitor cells are generated from a population of HLA-defined hiPSCs. In certain embodiments, the population of HLA-defined hiPSCs comprise human CD34+ cord blood cells.

In certain embodiments, the FF and XF culture of the hiPSCs in contact with the tankyrasc/PARP inhibitor-continuing composition comprises culturing the hiPSCs on vitronectin-coated substrates. In certain embodiments, the FF and XF culture of the hiPSCs in contact with the tankyrase/PARP inhibitor-containing composition is performed for 1to 10 passages. In certain embodiments, passages 1-3 of the FF and XF culture of the hiPSCs in contact with the tankyrase/PARP inhibitor-containing composition comprise selection of dome-shaped colonies for each subsequent passage.

### 4. BRIEF DESCRIPTION OF THE DRAWINGS

FIGS. 1A-1B. Patient-specific Naïve Progenitor Cell Therapies from a Defined cGMP-grade Bank of HLA-defined, Universal Donor Tankyrase/PARP inhibitor-regulated naïve human induced pluripotent stem cell (UTIRN-hiPSC) lines. (A) HLA-defined UTIRN-hiPSC can ultimately be more cost-effective than patient-specific strategies in serving the needs of larger numbers of patients requiring immediate, multi-lineage regeneration of complex diseased tissues. Although TIRN-hiPSC-derived cells are likely to generate allogeneic immune responses in non-autologous recipients, a bank of clinical-grade cGMP TIRN-hiPSC lines, HLA-matched to HLA A, B, and DR loci (and O-negative blood typed) would significantly expand the therapeutic feasibility of hiPSC and reduce graft rejection. (B) The Johns Hopkins post-transplant cyclophosphamide (Pt-Cy) haplo-identical bone marrow transplantation (BMT) prep regimens provide a versatile platform for generating immune tolerance to haplo-identical HLA-defined hiPSC ‘haplobanks’. HLA-tolerizing UTIRN-hiPSC-derived hematopoietic progenitors as well as a transplantable UTIRN-hiPSC progenitors of choice (e.g., cardiac, vascular, or retinal) could be differentiated from the same HLA-banked UTIRN-hiPSC line for induction of tissue transplantation tolerance using the PtCy method.

FIG. 2. Universal Donor Tissue Transplantation Tolerance with UTIRN-hiPSC and the Pt-Cy Method. FIG. 2 depicts a strategy for cost-effectively generating patient-specific progenitor cell therapies by generating a bank of HLA-defined, clinical-grade, ‘universal donor’ UTIRN-hiPSC lines. HLA-defined ‘universal donor’ UTIRN-hiPSC will ultimately be more cost-effective than patient-specific strategies, and can better serve the needs of larger numbers of patients requiring immediate, multi-lineage regeneration of complex diseased tissues. Computational models predict that a small number of HLA-defined hiPSC lines derived from existing HLA-typed cord and blood banks could generate matches to serve the transplantation needs of the majority of populations in Japan, UK, and the USA. To prioritize immune compatibility to as many individuals as possible, blood group O genotyped individuals from 2-10 homozygous HLA haplotype blood donors could serve the needs of 5-30% of the population. Although hiPSC-derived cells are likely to generate allogeneic immune responses in non-autologous recipients, a bank of clinical-grade cGMP hiPSC lines, HLA-matched to HLA A, B, and DR loci (and O-negative typed) would significantly expand the therapeutic feasibility of hiPSC and reduce graft rejection, especially if used along with an established hematopoietic tolerance induction protocol (e.g., PtCy).

FIGS. 3A-3D. Tankyrase/PARP inhibition promotes stable rewiring of conventional hPSC to a preimplantation naïve epiblast-like state with intact epigenomic imprints. The inclusion of the tankyrase/PARP inhibitor XAV939 to the classical LIF-2i method was sufficient for stable expansion of hPSC in a TIRN state on feeders, and was validated to stably naïve revert over 30 hPSC lines; independent of genetic donor background. (3A) (left panels) Three representative hPSC lines (H9 hESC, cord blood (CB)-hiPSC, fibroblast (fib)-hiPSC) are shown in their starting primed (i.e., Essential 8 (E8) medium) conditions, and 6-10 passages post culture in continuous LIF-3i naïve-reverted conditions. Monolayer bFGF-dependent primed hPSC colonies become tolerant to bulk single-cell passaging and acquire a typical dome-shape morphology. (middle panels) TIRN-hPSC retain strong expression of TRA-1-81 and SSEA4 surface antigens by flow cytometry. (right panels) Western blot analyses in these primed and TIRN-hPSC lines demonstrated that TIRN-hPSC acquired active phosphorylated STAT3and reduced ERK1/2 phosphorylation. (3B) LIF-3i-reverted TIRN-hPSC acquire defining human preimplantation epiblast molecular characteristics. Principal component analysis (PCA) of single-cell RNA-seq datasets from human pre-implantation embryos were normalized with transcriptome (gene array) datasets of 12 isogenic primed/TIRN-reverted pairs of genetically independent hPSC lines. While primed hPSC overlapped with late/pseudo-post-implantation E7 human epiblast cells, the bulk transcriptomic signatures of tankyrase/PARP-inhibited TIRN-hPSC uniformly shifted toward a pre-implantation identity at ˜ the human E6 epiblast developmental stage. Shown are first and second PCAs generated using the binary kTSP signature data matrix for Petropoulos, Zimmerlin, and PCBC gene expression data sets (see Methods FIG. 3, below). (3C) Western blot analysis of primed (P) and naïve (N) hPSC. TIRN-hiPSC (CB-hiPSC E5C3) markedly upregulated DNMT3L but maintained DNMT1 protein expressions. (3D) Infinium CpG methylation heatmap of genomic Imprinted regions of a panel of primed and naïve isogenic hPSC samples. Heatmap compares previously published Zambidis lab methylation data with published Court et al., Genome Res 24, 554-569, (2014) data of imprinted genomic regulatory regions (methylation beta values: 0—completely hypomethylated probe; 1—completely methylated probe). Probes are sorted by chromosomal location and arranged into their adjacent primed (−) and naïve (+) hPSC isogenic pairs. Zambidis lab methylation beta values were subset to exact imprinted regions provided by Court et al. The imprinted regions of Court et al.'s abnormal androgenetic hydatidiform mole and other control samples (e.g., sperm, normal human tissues, and control primed hESC) are also shown.

FIG. 4. Epigenetic model for chemical naïve reprogramming of conventional hiPSC with Tankyrase/PARP inhibition. Waddington landscape model for the epigenetic barriers posed by lineage priming, incomplete reprogramming, and disease-associated epigenetic aberrations in primed hiPSC (dashed line). These obstacles may be overcome with molecular reversion to a tankyrase/PARP inhibitor-regulated naïve epiblast-like state (solid line) possessing a developmentally naive epiblast-like epigenetic configuration.

FIGS. 5A-5D. Xeno-free (XF), Feeder-free (FF) primed cord blood (CB)-hiPSC generation. (5A) Reprogramming schematic. (5B) Morphology of XF/FF CB-hiPSC colonies during reprogramming, and following expansion (P1=1st passage after reprogramming; P8=8th passage). Shown in lower panels are confirmation of hiPSC phenotype using alkaline phosphatase activity assay and TRA-1-81 immunofluorescent staining. (5C) Reprogramming efficiencies (measured as the percentage of alkaline phosphatase (AP) activity positive colonies per reprogrammed cell) for xenofree (XF) reprogramming with or without stroma-priming (SP). Efficiency for non-XF reprogramming for matching CD34⁺ cells (non-XF) is shown as control. (5D) Expression of pluripotency-associated markers (i.e., SSEA4, TRA-1-81, NANOG, OCT4 and SOX2) in reprogrammed XF/FF CB-hiPSC. Isotype controls are shown to validate specificity of intracellular and extracellular FACS immunostainings.

FIGS. 6A-6E. Functional Pluripotency of XF/FF primed CB-hiPSC. (6A) Validation of XF-hiPSC multi-lineage functional pluripotency by teratoma assay. Five million cells were subcutaneously injected into the limb of NSG immunodeficient mice. Eight weeks later the teratoma tissue was excised, fixed in formaldehyde and embedded in paraffin. A representative teratoma microsection microsection is shown after staining with eosin and hematoxylin. XF-hiPSC lines produced matured tissues in teratoma with closed juxtaposition of lineages from all three germ layers (ectoderm (ecto), endoderm (endo), mesoderm (meso)). (6B, 6C) Directed differentiation of XF/FF CB-hiPSC toward hematopoietic lineages. Hematopoietic directed differentiations of two XF/FF CB-hiPSC lines were compared to a non-XF CB-iPSC line (i.e., 6.2 hiPSC). CB-hiPSC were differentiated for 8 days using an established embryoid body (EB) protocol. EB were digested using collagenase type IV (1 mg/mL) and dissociated cells were transferred onto fibronectin-coated plates (10 ug/mL) and cultured in endothelial growth medium (complete EGM2 (Lonza) supplemented with 25 ng/mL VEGF). The medium was replaced the next day and after 2 additional days. Floating CD34+ progenitors were collected and transferred into methylcellulose medium (MethoCult, StemCell Technologies) for hematopoietic CFU assay. Representative photomicrographs (6B) of CFU generated from XF/XF CB-hiPSC are shown. (6C) The number of crythroid and myeloid CFU were counted 17 days after depositing 100,000 EB-derived cells for control CB-hiPSC control line 6.2 and two XF/FF-CB-hiPSC lines. (6D, 6E) Directed differentiation of XF/FF CB-hiPSC toward definitive endoderm. Definitive endoderm directed differentiations of two XF/FF CB-hiPSC lines were compared to a non-XF CB-iPSC line (i.e., H9 hESC) using a commercially available differentiation kit (StemDiff, StemCell Technologies). Representative photomicrographs of day-7 endodermal differentiations are shown (6D) with corresponding FACS analysis of CXC4 and SOX17(6E).

FIGS. 7A-7C. Stepwise transition of XF/FF primed hPSC cultures to FF/XF LIF-4i Tankyrase/PARP inhibitor-regulated naïve (TIRN)-hiPSC conditions. (7A) Schematic of the stepwise LIF-4i FF TIRN reversion protocol. (7B) Representative photomicrographs of hPSC morphology during LIF-4i FF reversion using commercially available human episomal iPSC line 6.2 (ThermoFisher #A18945) derived in the Zambidis lab. The transition between conventional flat monolayer primed colonies and dome-shaped clonogenic LIF-4i FF cultures is shown. Overnight supplementation of 3-to-4-day-old E8cultures with 10% Knock-Out serum can improve initial clonal recovery for subsequent bulk reversion and expansion in LIF-4i FF. For most cell lines, this strategy does not require colony picking. The use of anti-apoptotic molecules (i.e. ROCK inhibitor Y-27632.2-5 uM) is recommended for overnight post-passaging. We have successfully employed this strategy to expand a series of genetically independent LIF-4i FF hPSC cultures (n=10: H9, RUES1, RUES2, RUES3 hESC; E5C3, 6.2 non-XF CB-iPSC (i.e., Gibco® Cat #A18945); E32C1XF, E32C3XF; E32C6XF XF CB-iPSC, C2 adult skin fibroblast-hiPSC) using non-enzymatic dissociation methods (e.g., EDTA-based Versene or Gibco® enzyme-free cell dissociation buffer) without detecting any chromosomal abnormality, including at high passages (e.g., up to P40). Scale bars=200 μm. (7C) Flow cytometry analysis of the expression of the extra-cellular antigens SSEA-4 and TRA-1-81 in the human iPSC line 6.2 at successive passages in the LIF-4i system.

FIGS. 8A-8C. Stability of LIF-4i of feeder-free culture conditions is dependent on Tankyrase/PARP (XAV939) inhibition but reinforced by PKC inhibition (Go6983). (8A) Retention of the TRA-1-81⁺SSEA4⁺ phenotype by FACS in LIF-4i TIRN-hPSC is shown as baseline for representative hiPSC (E5C3, red) and hESC (H9, orange) lines. Alternatively, E5C3 CB-hiPSC were maintained in LIF-4i for 3passages (6 days) and then cither XAV939 (E5C3-XAV939, square datapoints, dotted line) or Go6983 (E5C3 LIF-3i, triangles, dashed line) were removed from the LIF-4i formulation. XAV939 removal induced rapid loss of TRA-1-81 and SSEA4 expression. Because reduced levels of CHIR99021 (1 uM) have been described to reduce CHIR99021-induced differentiation effects in the presence of PD0325901 MEK inhibitor and Go6983in another naïve culture system⁴⁶, whether reduced CHIR99021 concentration could rescue XAV939 depletion (E5C3—XAV939, CHIR99021 @ 1 uM) was evaluated, but destabilization of LIF-4i naïve pluripotency in absence of XAV939 was not rescued by lower concentration of CHIR99021. While the differentiating effects of Go6983 exclusion from LIF-4i were not as dramatic as XAV939 removal, hiPSC started differentiating after a few days without Go6983. (8B) Representative photomicrographs and flow cytometry plots of CB-hiPSC when continuously cultured in LIF-4i (LIF+4i, 6 passages), or when cultured in LIF-4i for 3 passages and then transferred into FF conditions that omitted for 3 passages the PKC inhibitor Go6983 (LIF+3i) or the tankyrase/PARP inhibitor XAV939 (without XAV939) or without XAV939 and reducing the concentration of CHIR99021 (without XAV939, CHIR 1 uM). Loss of TRA-1-81⁺SSEA4⁺ was accompanied with loss of undifferentiated dome-shaped colonies and the emergence of a monolayer of differentiated cells. (8C) Photomicrographs and flow cytometry plots demonstrating retention of the TRA-1-81⁺ SSEA4⁺ phenotype in established TIRN-hESC (H9) and representative TIRN-hiPSC lines in LIF-4i TIRN conditions.

FIGS. 9A-9C. Optimization of the transition step from E8 primed hPSC conditions to LIF-4i TIRN conditions: clonal cell passaging. (9A) Clonal efficiency during the first passage in LIF-4i TIRN conditions for hESC line (RUES01). Supplementation of LIF-3i with forskolin and purmophamine (i.e., LIF-5i) and brief adaptation of primed hPSC was previously shown to facilitate clonal transition and survival of primed cells to TIRN hPSC when subsequently cultured continuously in LIF-3i TIRN conditions on feeders. However, we found that prolonged exposure to LIF-5i of primed hPSC for several days in the first passage without feeders promoted spontancous differentiation. Therefore, the brief LIF-5i adaptation period was limited to only overnight exposure prior to passaging. However, adaptation of E8 primed media with 10% KOSR facilitated initial clonal efficiencies in following LIF-4i TIRN reversion. (9A—left panel) Starting from early passage E8 hPSC cultures at low density also dramatically improved clonal efficiencies for reversion of primed RUES01 hESC following 2 passages in LIF-4i TIRN conditions. (9A—middle, right panels) In contrast, high density culture in E8 (e.g., up to 6 days) prior to passaging in LIF-4i negatively impacted the transition into LIF-4i TIRN conditions. Photomicrographs represent primed cultures immediately prior to passaging into LIF-4i TIRN conditions. (9B) Requirement for ROCK inhibitor Y27632during passaging. Overnight exposure to low dose of Y-27632 (2-5 uM) is sufficient and required to promote efficient attachment of LIF-4i UTIRN-CB-hiPSC (E32C6XF) during passaging onto Vitronectin-XF-coated plates.

FIGS. 10A-10B. Optimization of the transition step from E8 primed hPSC conditions to LIF-4i TIRN conditions: detection of differentiated lineages and undifferentiated PSC after 2 passages in LIF-4i TIRN conditions. Primed hPSC are characterized by pronounced interline variability of differentiation. While some hPSC lines do not produce any differentiated lineages during LIF-4i reversion, most initially generate simultaneously undifferentiated naïve colonies and various differentiated lineages, which will usually only emerge during the second passage in LIF-4i. Undifferentiated colonies can be bulk passaged using differential sensitivity to EDTA-based non-enzymatic dissociation buffers (e.g., Versene or Gibco® enzyme-free cell dissociation buffer). (10A) Concomitant detection of undifferentiated OCT4+colonies and differentiated lineages (e.g., GATA4^{+,} PAX6⁺ or cytokeratin 8⁺ cells) in representative hPSC after 2 passages in LIF-4i by immunofluorescent microscopy. (10B) Using non-enzymatic dissociation buffers (e.g., Versene), undifferentiated colonies can be lifted and collected while differentiated cells remain attached to the plate. Residual cells are shown after a 5-minute exposure to the dissociation buffer.

FIG. 11. G-banding Karyotypes of primed and LIF-4i-reverted XF/FF hiPSC. Karyotype analysis was performed by the JHU Cytogenetics Core Facility. Normal G-banding karyotypes were confirmed in primed E8 male and female XF/FF UTIRN-CB-hiPSC (E32C6XF, E32C1XF) and control hESC (RUES1) cultures before LIF-4i TIRN reversion, and then in stable LIF-4i TIRN cultures at various passages (up to 40 passages in LIF-4i).

FIGS. 12A-12C. Expression of naïve epiblast-specific transcripts in XF/FF UTIRN-hiPSC (qRT-PCR). Expression of naïve epiblast-specific transcripts and retroelements associated with early pre-implantation embryo cells were assayed via qRT-PCR following XF/FF LIF-4i TIRN reversion of primed hPSC. Results are shown between isogenic hPSC cultures that were maintained in parallel E8 (primed) and FF LIF-4i (TIRN) states. For all experiments, 1 μg of isolated RNA was processed using the SuperScript VILO cDNA Synthesis Kit (Life Technologies) using a MasterCycler EPgradient (Eppendorf) prior to being used for PCR analysis using the TaqMan Fast Advanced Master Mix (Life Technologies) and Taqman gene expression assays (Life Technologies). For all experiments, beta-actin was employed as a reference gene. (12A) Relative quantification of naïve epiblast-specific genes and early pre-implantation embryo-associated retroviral element expressions in the established hESC line H9 when cultured in FF naïve (LIF-4i) medium compared with isogenic parallel maintenance in E8 medium. H9 cells that were cultured in LIF-4i displayed enhanced expression of early naïve markers (i.e., KLF2, ZSCAN4) with robust expression of KLF17 as well as marked increase of expression of endogenous retroviruses HERV-H and HERV-K at specific loci that were detected using Taqman assays. These effects appear to only target specific retroviral sequences (i.e., HERV-H and HERV-K) that have been shown to mark early naïve subsets in rodents and primates, since other retroviral elements were downregulated (e.g., HERV-FRD, HERV-W) and no increased expression for some specific HERV-H loci (e.g., ERVH-1, ERVH-6) was detected. (12B) Relative quantification of naïve epiblast marker expression in hESC H9 and non-XF CB-hiPSC E5C3 lines. Increased expression of genes associated with naïve pluripotency (KLF2, NR5A2, DNMT3L, STELLA (DPPA3)) is reproducible using TIRN-hiPSC lines with distinct genetic backgrounds. (12C) Validation of gene expression changes in LIF-4i for XF/FF UTIRN CB-hiPSC cells lines and the hESC control line RUES01. Upregulation of naïve genes observed in TIRN-reverted cell lines (e.g., hESC H9 and hiPSC E5C3) not originally derived in XF/FF conditions was similar to XF/FF UTIRN CB-hiPSC lines.

FIGS. 13A-13B. Expression of naïve epiblast-specific factors and proteins in primed vs XF/FF UTIRN-hiPSC with immunofluorescence (IF) microscopy. (13A) Comparison of protein expression of pluripotency factors by IF in the CB-hiPSC cell line E5C3 in E8 (primed) vs LIF-4i (TIRN) conditions. LIF-4i medium promoted uniform retention of expression of the pluripotency factors NANOG, SOX2 and OCT4. Unlike primed E5C3 cells in E8, LIF-4i TIRN-hPSC cultures acquired homogenous protein expression of STELLA (DPPA3), TFCP2L1, CD77 and E-Cadherin. The naïve markers KLF17 and DNMT3L were strongly upregulated in a subset of LIF4i cells. While the histone repressive mark H3K27me3 was detected at strong levels within foci in primed E8 cells, it displayed a diffused pattern with lower intensity in LIF-4i colonies. (13B) Validation of expression of pluripotency factors in XF/FF-derived HLA-predefined universal UTIRN-CB-hiPSC line E32C4XF. XF/FF-adapted TIRN-CB-hiPSC E5C3 retained uniform expression of core pluripotency factors NANOG and OCT4, and acquired expression of naïve markers (e.g., NR5A2). As previously established, the LIF-3i/MEF method promoted upregulation of active beta-catenin in TIRN hiPSC, especially in non-nuclear compartments, and we confirmed a similar result following TIRN reversion of primed XF/FF E32C4XF CB-hiPSC following continuous culture in LIF-4i. (13C) Alkaline phosphatase staining of LIF-4i-reverted TIRN-CB-hiPSC.

FIG. 14. Molecular characterization of primed vs. XF/FF UTIRN-CB-hiPSC. Primed (E8) vs. TIRN (LIF-4i) phosphorylated (phospho-STAT3) expression at designated passages post TIRN reversion. Western blots were performed of primed vs. TIRN lysates of hESC RUES02 and three independent XF/FF UTIRN CB-hiPSC lines (E32C1XF, E32C4XF, E32C6XF). ACTIN, HSP90 and total STAT3 served as internal loading controls. Western blots of XAV939-inhibited proteolysis of tankyrases 1 and 2(TANK 1/2) and AXIN-1 proteins in isogenic primed vs. TIRN conditions for TIRN-reverted RUES02 and XF/FF CB-hiPSC lines. Western blot analysis of hPSC lines in primed (E8) and naïve (LIF-4i) cultures for expression of phosphorylated (phospho.) STAT3 and ERK1/2, tankyrase 1/2 and axin1. LIF-4i cultures displayed elevated phosphorylated levels of STAT3 with concomitant decrease of ERK 1/2 phosphorylation. Multiple proteins that are regulated by tankyrase-mediated poly-ADP-ribosylation (e.g., AXIN1 and tankyrases) were upregulated due to inhibiting PARylation activities of tankyrase, as well as other non-tankyrase PARP activities; since XAV939 has broad PARP inhibition activities.

FIGS. 15A-15C. Activities of proximal enhancer (PE) and distal enhancer (DE) elements of the hOCT4 promoter in primed (10 ng/mL bFGF) vs. LIF-4i TIRN-reverted hiPSC. Fluorescent microscopy detection of GFP and cytometry plots of representative LIF-4i TIRN-reverted hiPSC subclones of the Fibroblast-hiPSC C2 cell line that were stably transfected with full-length OCT4-GFP-2A-PURO PE/DE sequences (15A), mutant DPE-OCT4-GFP-PURO and DDE-OCT4-GFP-PURO constructs (15B, 15C), and non-transfected controls (i.e., no construct, primed control, TIRN control). Shown are % GFP⁺ cells detected by FACS within undifferentiated TRA-1-60+SSEA4+ cells for individual hiPSC subclones expressing control or mutant DPE sequences that were maintained in FF primed (supplemented with 10 ng/ml bFGF), or after short exposure (2 passages, 4 days) to LIF-4i TIRN cultures.

FIGS. 16A-16C. Comparison of functional pluripotency between isogenic FF primed (E8) and XF/FF TIRN (LIF-4i) states: Teratoma studies of XF/FF UTIRN-CB-hiPSC. (16A). Schematic of strategy for validating functional pluripotency of E8 primed vs LIF-4i TIRN pluripotent states in comparative teratoma assays. (16B). Isogenic FF conventional, primed (E8) and FF UTIRN hiPSC (LIF-4i, 10 passages) E32C6XF lines were injected subcutaneously (5×10⁶ cells per site) into NSG immunodeficient mice. Eight weeks later, teratomas were simultaneously recovered from injected animals. Macroscopic and microscopic observation revealed striking differences between isogenic primed (E8) hPSC-derived and TIRN HPSC-derived teratomas. Primed teratomas displayed a typical and significant bias toward definitive endoderm lineages with observable cysts and pigmented epithelium. (16C). Hematoxylin and eosin staining of teratoma sections were performed to evaluate differentiation into lineages of the three germ layers (ectoderm (ecto), mesoderm (meso), and endoderm (endo)) from primed/E8 and LIF-4i UTIRN-hiPSC. Primed culture of the hiPSC line E32C6XF did not produce clearly identifiable neuro-ectoderm tissues. In contrast XF/FF UTIRN-hiPSC-derived teratomas generated significantly more robust and numerous areas of neural rosettes and pigmented epithelium. Similarly, while only limited mesodermal areas were detected in teratomas that were generated from primed cells, extensive and robust chondroblast and osteoblast formation was detected in XF/FF UTIRN-hiPSC teratoma sections. Finally, both conditions produced well-differentiated definitive endoderm lineages with highly cystic areas. All scale bars in this figure are 100 um.

FIGS. 17A-17C. Comparison of functional pluripotency between isogenic FF primed (E8) and FF TIRN (LIF-4i) states: Hematovascular differentiation of XF/FF TIRN-CB-hiPSC. (17A) Schematic strategy for assessing functional pluripotency from E8 and LIF-4i TIRN pluripotent states via hematovascular differentiation. (17B, 17C) APEL monolayer hematovascular differentiation system). APEL monolayer differentiation of primed (Blue hatched) vs LIF-4i FF TIRN-hPSC (red hatched). Representative results are shown for the CB-derived hiPSC line 6.2(ThermoFisher #A18945) using the XF monolayer APEL differentiation system, which is versatile and can be adapted to generate hematopoietic, endothelial or perivascular lineages. XF/FF UTIRN-CB-hiPSC robustly differentiated with improved generation of CD31⁺CD34⁺CD144⁺ vascular cells using the XF APEL mesodermal differentiation system (without a re-priming step). Vascular directed differentiation (e.g., supplementation of APEL medium with VEGF and TGF-beta inhibition) of UTIRN-CB-hiPSC generated robust and higher levels of CD34+ progenitors relative to isogenic primed E8 control. (17C) Pericytic Vascular progenitors (VP) were differentiated from the CB-hiPSC line 6.2 in APEL medium for 10 days, MACS-sorted for CD31⁺ endothelial expression and expanded in endothelial growth medium (EGM2, Lonza) onto fibronectin for 2 passages. While VP generated from UTIRN-CB-hiPSC retained homogenous expression of CD31, with most VP also co-expressing the proliferation-associated marker Ki67, CD31⁺ VP that were produced from isogenic primed cells poorly expressed CD31 and Ki67⁺ proliferative cells. LDL uptake assays of VP generated from primed vs TIRN-hiPSC (6.2 CB-hiPSC) were performed at the first passage (9 days of culture in EGM2) to assess their vascular functionality.

FIGS. 18A-18D. Comparison of vascular differentiation kinetics between isogenic FF primed (E8) and FF/XF TIRN (LIF-4i) hiPSC states. (18A) Schematic for strategy of assessing functional pluripotency from E8 and TIRN pluripotent states via APEL vascular differentiation. Note that for efficient differentiation of TIRN-hiPSC, the PKC inhibitor Go6983 must be omitted for at least 1 hour prior to differentiating into APEL. (18B) Isogenic comparisons of three genetically independent cell lines: RUES01 hESC, and two CB-hiPSC lines (6.2, E5C3): emergence of vascular lineages in APEL differentiation when starting in primed (E8) and XF/FF TIRN LIF-4i cultures. FACS plots are shown for CD34 (hematovascular lineages) and CD140b (perivascular lineages) expression after 4 days of differentiation in APEL medium. Following mesodermal determination and early specification, all 3 cell lines showed more robust generation of CD34⁺ vascular and CD140b⁺ perivascular progenitors when starting from LIF-4i compared with simultaneous isogenic cultures in E8. (18C) Isogenic comparisons of vascular lineage specification in APEL medium. VP differentiations were directed toward endothelial lineages using VEGF supplementation. Primed hPSC cultures display pronounced variability in differentiation capacity. FACS plots are shown for day-10 vascular differentiations of three genetically independent primed cultures and parallel LIF-4i naïve cultures. After 10 days of differentiation in APEL, isogenic parallel differentiations of TIRN-hiPSC cultures generated higher frequencies of CD34⁺ progenitors with limited non-directed perivascular bias compared to primed cells. (18D) FACS plots for Day-4 APEL differentiations of a representative cell line (CB-hiPSC E5C3) demonstrated improved vascular generation from TIRN-hiPSC compared to primed conditions (e.g., Embryonic VP expressing CD31+CD146+ cells and co-expressing KDR, CD73, CD105 (endoglin) and CD144.

FIGS. 19A-19D. Western Blot analysis of NHEJ and HDR proteins in primed (E8) hPSC and HLA-defined UTIRN-hPSC. The XF/FF-derived CB-hiPSC line E32C6XF was reverted into UTIRN-hiPSC using the XF/FF LIF-4i method and expanded for 10 passages. Cell lysates were prepared from parallel primed (E8) and TIRN (LIF-4i) cultures and 20 or 35 ug of proteins were loaded per lane. Both primed and TIRN-reverted hiPSC expressed significant amounts of DNA-PKc, the catalytic subunit of DNA-PK. which is a critical component of the NHEJ machinery for repairing DNA double strand breaks. DNA-PKc PARylation by PARP1/2 and tankyrases regulates DNA PK kinase activity in the DNA damage response. PCNA levels are shown as a chromatin-bound protein loading control. (19A, 19B) After 10 passages in LIF-4i medium, UTIRN-hPSC expressed lower amounts of total (T) and phosphorylated (P) H2AX, indicating lower levels of DNA damage in culture. As reported for the LIF-3i/MEF method, we detected increased expression levels of tankyrase 1 and 2 proteins (TANK1/2), due to XAV939-mediated inhibition of tankyrase self-PARylation and subsequent associated ubiquitin-mediated proteolysis in proteosomes. Accumulation of TANK1/2 in UTIRN-hPSC correlated with stabilization of its binding partner, the DNA damage sensor MDC1. MDC1 is a DNA checkpoint protein that is essential to activating the DNA response machinery and that is cleaved by caspase3 during apoptosis. MDC1 cleavage was reduced in the LIF-4i culture. Tankyrase-mediated activation of homologous recombination DNA repair machinery has been shown to involve MDC1 and BRCA1 complexes. This tankyrase activity is independent of tankyrase-mediated PARylation, and was not diminished in the presence of the tankyrasc/PARP inhibitor XAV939 in the 293T cell line. Similarly, we detected increased levels of non-cleaved MDC1 in XF/FF UTIRNCB-hiPSC, as well as increased levels of BRCA1. While UTIRN-hiPSC displayed lower levels of RAD51, they endogenously maintained expression of RAD54. (19C, 19D) Effects of LIF-4i on the DNA repair machinery were reproducible in multiple XF/FF UTIRN-CB-hiPSC lines (E32C1XF, 10 passages in LIF-4i, E32C4XF,10 passages in LIF-4i, E32C6XF, 14 passages in LIF-4i). Accumulation of tankyrase protein in LIF-4i TIRN cultures correlated with lower levels of total and phosphorylated H2AX and cleaved MDC1, indicating lower detection of double strand breaks and apoptosis in LIF-4i. Inhibition of PARP1-mediated PARylation of DNA-PKc promote DNAPK auto-phosphorylation and activation. Interestingly, XF/FF UTIRN-CB-hiPSC lines displayed reinforced levels of phospho-DNA-PKc (19D) in agreement with the observations that the broad and promiscuous tankyrasc/PARP inhibitor XAV939 inhibits not only tankyrase activities (PARP 5a/b), but also downregulates PARP1-mediated PARylation activities. XF/FF UTIRN-CB-hiPSC also upregulated BRCA1 and variable levels of RAD51 and RAD54. Activation of these DNA double strand repair proteins may contribute to lower amounts of H2AX protein.

FIGS. 20A-20B. Induction of DNA damage in primed hPSC and XF/FF UTIRN-hPSC following exposure to the radiomimetic DNA damage inducing agent neocarzinostatin (NCS). Unlike somatic cells, naïve mouse embryonic stem cells (ESC) employ homologous recombination machinery to repair double strand breaks. However, imbalanced levels of DNAPKc in mouse ESC results into poor capacity for rejoining of radiation-induced DNA double-strand breaks in comparison to human primed ESC. Interestingly, XF/FF UTIRN-CB-hiPSC and TIRN-H9-hESC responded robustly to the radiomimetic NCS treatment and accumulated levels of DNA repair machinery including (20A) P-H2AX and (20B) p-DNA-PKc and RAD54 that were superior to primed E8controls. Superior activation of the DNA repair machinery in NCS-treated UTIRN-hiPSC also correlated with lower Caspase 3 and RAD54 expression than primed hPSC, thus supporting the hypothesis that TIRN-hPSC tolerated and were more efficient in repairing NCS-induced DNA damage-associated breaks (n=3 independent hPSC lines).

FIG. 21. Summary schematic for the advantages and future utility of UTIRN-hiPSC in Regenerative Medicine.

### 5. DETAILED DESCRIPTION

The present disclosure relates to methods for generating both patient-specific and universal donor banked HLA-defined therapeutic progenitors in clinical grade, current good manufacturing practice (cGMP)-compliant conditions. In certain embodiments, the patient-specific and the universal donor banked HLA-defined therapeutic progenitors are derived from a new class of tankyrase/PARP inhibitor-regulated naïve human induced pluripotent stem cells (TIRN-hiPSCs).

In one aspect, the present disclosure is directed to methods and compositions finding use in the production of patient-specific TIRN-hiPSCs. For example, patient-specific TIRN-hiPSCs can be derived from any human somatic cell using the methods described herein. These patient-specific TIRN-hiPSCs represent a new class of human naïve pluripotent stem cells exhibiting high epigenetic plasticity, stable epigenomic imprints, and more efficient multi-lineage functionality than conventional, lineage-primed hiPSC. The methods described herein result in efficient, bulk, and rapid chemical reversion of conventional, lineage-primed, xenofree (XF) feeder-free (FF) hiPSC lines into a stable preimplantation naïve epiblast-like pluripotent state in cGMP-grade conditions. For example, but not limitation, the chemical tankyrase/PARP inhibitor-based methodologies described herein rapidly revert conventional, lineage-primed hiPSC lines to adopt transcriptional, epigenetic, and biochemical features of the human pre-implantation naïve epiblast. Thus, in certain embodiments, the TIRN-hPSCs described herein possess multiple naïve ICM characteristics, including MEK-ERK/bFGF signaling independence, activated phosphorylated JAK/STAT3 signaling, distal OCT4 enhancer usage, global DNA CpG hypomethylation, and/or increased expression of activated beta-catenin. In certain embodiments, TIRN-hPSCs do not require reversion culture back to primed culture conditions prior to differentiation. In certain embodiments, reversion of conventional hiPSCs into TIRN-hiPSCs results in decreased lineage-primed gene expression and marked improvement in directed multi-lineage differentiation of conventional hiPSC lines across a broad repertoire of genetically-independent somatic cell hiPSC donors. In addition, TIRN-hPSCs can be protected against erosion of CpG methylated genomic imprinted regions and, in certain embodiments, maintain DNMT1 expression.

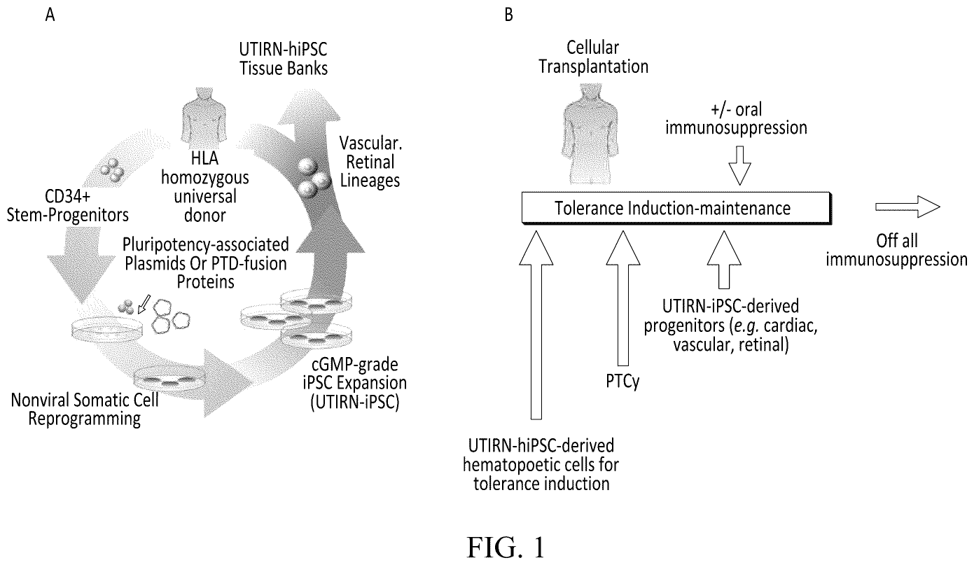

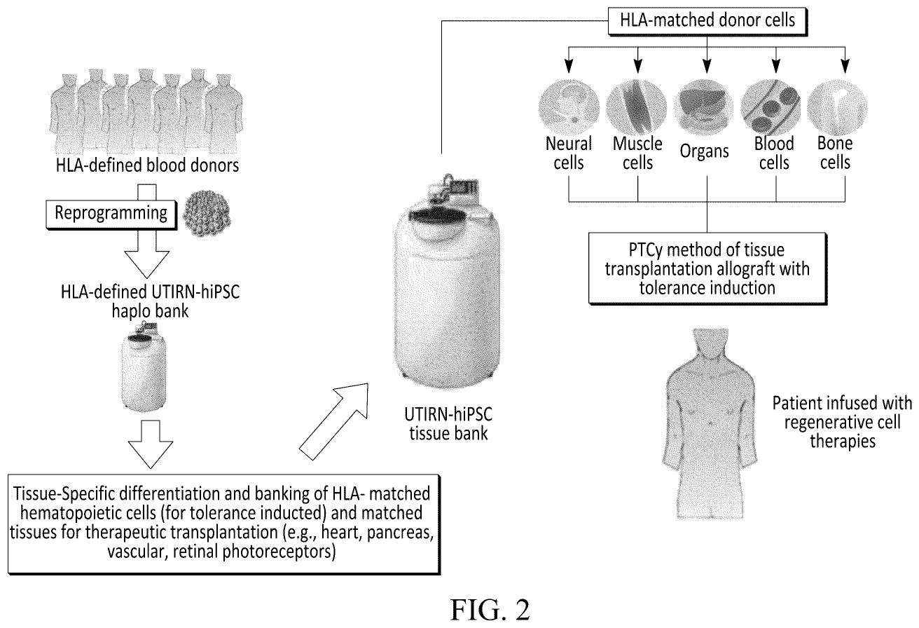

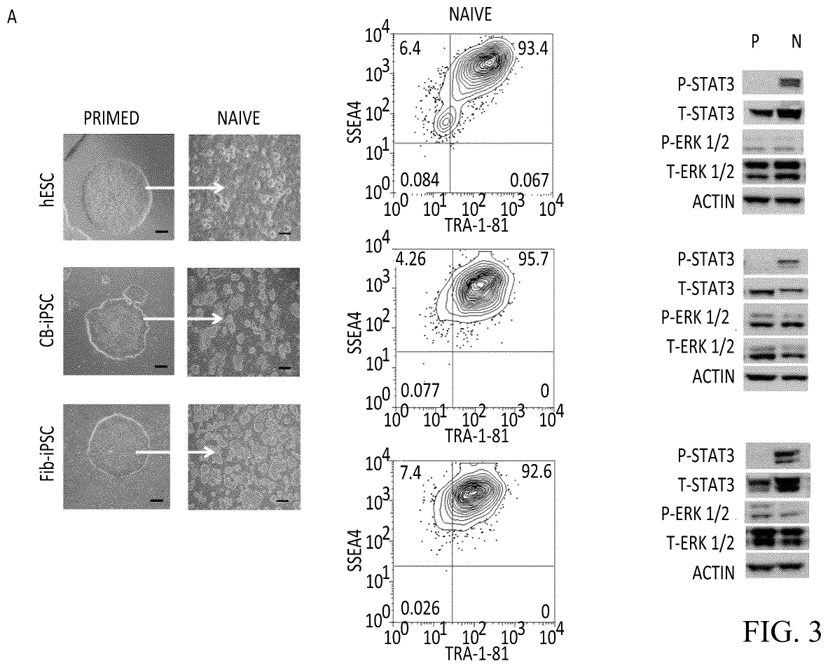

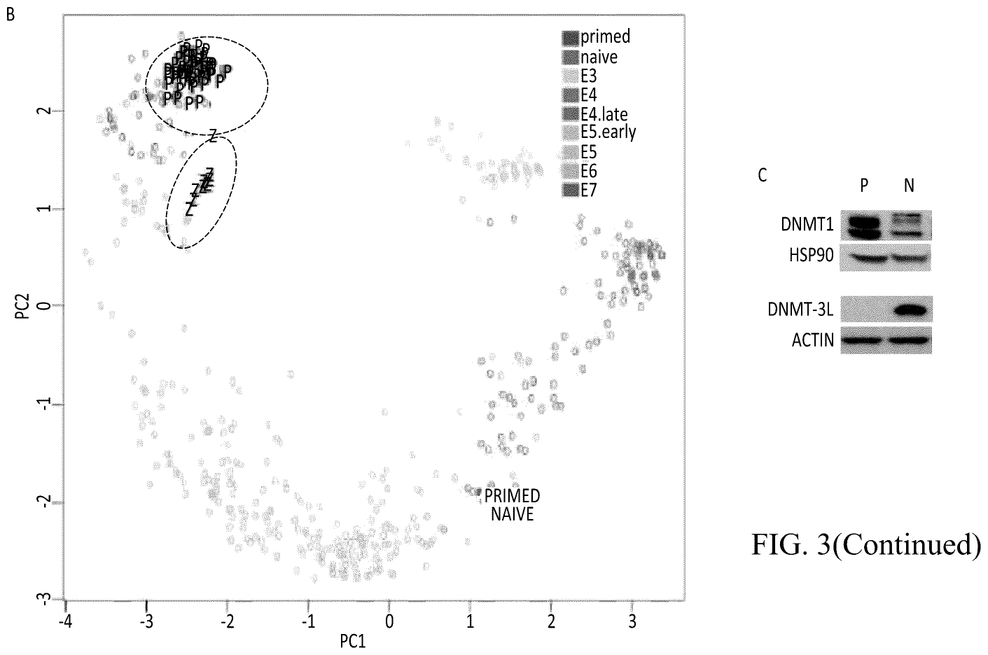

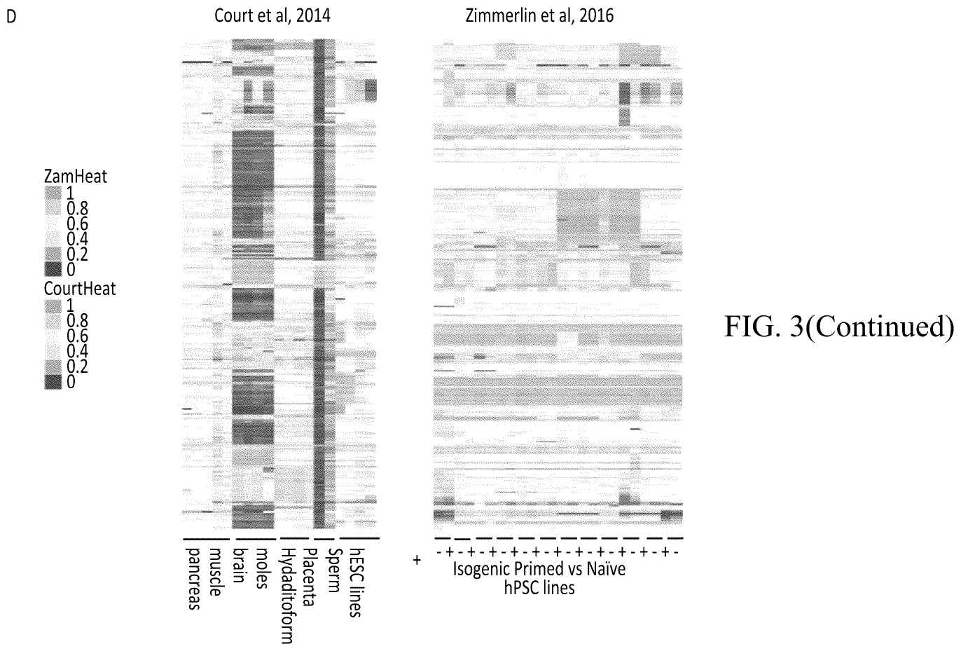

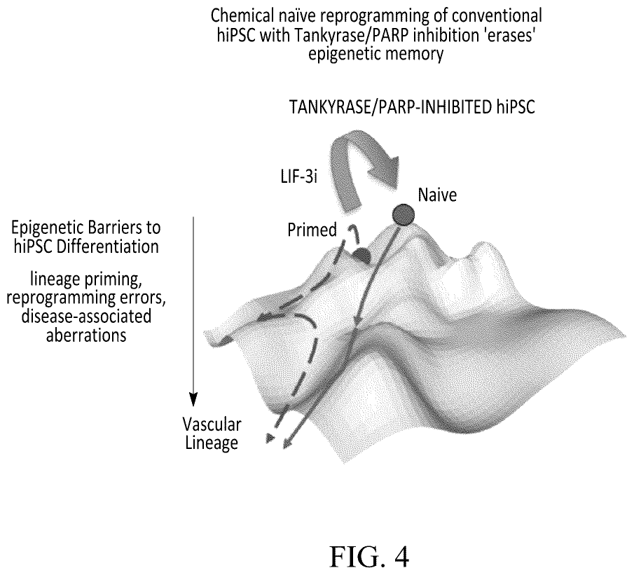

In another aspect, the present disclosure is directed to methods and compositions for establishing cGMP-grade banks of clinical grade ‘Universal’ donor TIRN-hiPSCs (UTIRN-hiPSCs) from HLA-defined CD34+ hematopoietic progenitors. These hematopoietic progenitor-derived UTIRN-hiPSCs can be utilized, for example, but not limitation, for comprehensive multi-lineage repair of diseased tissues in any recipient via approaches employing drug-mediated allogeneic tissue transplantation tolerance (e.g., with post-transplant cyclophosphamide). In certain embodiments, banks of UTIRN-hiPSC lines can be used to generate downstream, secondary banks of differentiated, HLA-defined, cryo-preserved universal donor cells (e.g., cardiac, vascular, neural progenitors) for “off-the-shelf” cellular therapies. In certain embodiments, UTIRN-hiPSCs will have high impact for regenerative medicine by facilitating allogeneic tissue tolerance induction strategies with a universal supply of cellular therapies in a manner that decreases the costs and broadens availability of hiPSC therapies to a wider number of individuals (e.g., as illustrated in FIG. 1 and FIG. 2).

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate equivalents thereof—and that other elements can be included and still fall within the scope/definition of the defined item, composition, apparatus, method, process, system, etc.

An “effective amount” as used herein, means an amount which provides a therapeutic or prophylactic benefit.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4,from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### 5.2. Production of Patient-Specific cGMP-Grade Banks of TIRN-hiPSCs

In one aspect, the subject matter of the present disclosure is directed to defined feeder-free (FF), xenofree (XF) cGMP-compliant culture medium systems for the production of patient-specific TIRN-hiPSCs. For example, the methods described herein are directed, in certain embodiments, to defined FF/XF cGMP-compliant culture medium systems for CB (or PBSC or BM) pluripotency episomal reprogramming. In certain embodiments, the methods described herein employ FF/XF medium systems that revert conventional XF/FF human induced pluripotent stem cells (hiPSCs) and human embryonic stem cells (hESC) to a naïve epiblast-like state, referred alternatively herein as the “LIF-4i FF TIRN system”, the “LIF-4i XF/FF TIRN system”, the “LIF-4i FF method”, the “LIF-4i XF/FF method” and modifications thereof. The LIF-4i FF TIRN system is based on a tankyrasc/PARP inhibitor-based small molecule approach. The method contains a novel recipe of LIF, four proprietary small molecules including a XAV939 (LIF-4i), defined media components, and KnockOut serum replacer (KSOR, e.g., Knockout™ Serum Replacement (Knockout™ SR) from Thermo Fisher), but does not contain growth factors typically required to maintain primed, conventional hiPSC (e.g., bFGF or TGFβ).

The LIF-4i FF TIRN system involves a modified version of the classic murine 2i naïve reversion cocktail to human pluripotent stem cells. For example, but not limitation, the self-renewal of hPSC (which cannot expand in 2i alone) is stabilized in LIF-2i by supplementing this cocktail with the tankyrase/PARP inhibitor XAV939. Although the mechanisms of action of XAV939 in hPSC are likely complex and synergistic with 2i, it likely includes at a minimum, an important stabilization and augmentation of hPSC self-renewal via WNT signaling pathways.

Furthermore, although the promiscuous PARP inhibitor XAV939 has been widely employed as a small molecule WNT inhibitor when used alone, another method, the LIF-3i method, accentuated several other studies that also demonstrated that when used simultaneously with GSK-3b inhibition, XAV939 induced a stabilization of AXIN isoforms that enhanced self-renewal of both conventional human pluripotent stem cells and murine EpiSC. The mechanism of action was shown to be XAV939 synergizing with GSK-3b inhibition, to paradoxically augment canonical WNT signaling by reinforcing the stability of the active isoform of β-catenin in both cytoplasmic and nuclear subcellular compartments. However, unlike previous studies, Zimmerlin et al., Development 2016; 143(22): 4368-4380, exploited dual use of GSK-3b/tankyrase/PARP co-inhibition along with simultaneous (PD0325901) MEK inhibition to stably revert a wide repertoire of conventional hPSC to a naïve epiblast-like state without requirement for exogeneous FGF2. In that study, initial culture in LIF-5i (including forskolin and purmorphamine) followed by LIF-3i culture reverted hPSC (TIRN-hPSC) re-activated naïve epiblast-like STAT3 signaling, and possessed naïve epiblast-like globally hypomethylated genomes, but without hypomethylated genomic imprinting aberrations. TIRN-hPSC were functionally competent for multi-lineage differentiation without need for an additional ‘capacitation’ step or re-culture back to primed state (i.e., ‘re-priming’). Moreover, these studies not only validated the functional pluripotency of reverted TIRN-hPSC in multi-lineage directed differentiation assays, but also revealed that TIRN-hPSC possessed significantly improved differentiation efficiencies relative to their isogenic primed, conventional hPSC counterparts.

The LIF-4i FF TIRN reversion method described herein is reproducible in a broad variety of independent FF hESC and transgene-free, non-integrated FF hiPSC lines. The method requires minimal training with basic cell culture skill and has been employed to revert >20 independent hESC and hiPSC lines from a broad array of donors. Furthermore, the LIF-4i FF TIRN system supports robust bulk clonal expansion efficiencies throughout all the steps between lineage-primed conventional hPSC culture all the way to completed naïve-like hPSC reversion (i.e., adaptation, transition and expansion for 7-10 passages in LIF-4i FF alone). The stability of this culture system does not depend on the presence of feeders and allows complete FF/XF expansion of TIRN-hPSC.

TABLE 1, below, outlines a two-step LIF-4i XF/FF TIRN method developed following extensive media analysis of the LIF-3i/MEF protocol. The LIF-4i FF chemical naïve reversion method employs the classical leukemia inhibitory factor (LIF), GSK3B, and MEK/ERK inhibition cocktail (LIF-2i), supplemented with only the tankyrase/PARP inhibitor XAV939 in a novel completely FF protocol. To stabilize TIRN-hPSC without feeders, the substrate and media composition were modified from the original LIF-3i/MEF method, and a 4th inhibitor (i.e., Go6983) targeting protein kinase C (LIF-4i FF medium) was introduced, which had been shown to reinforce pluripotency in human primed and rodent ESC. This mechanism is independent from STAT3 activation and MEK/GSK3β inhibition and antagonizes lineage commitment. This inhibitor has previously been introduced into other human naive culture systems to minimize spontaneous differentiation.

TABLE 1
LIF-4i XF/FF TIRN Method

| | Component | Exemplary Source(s) | Catalog No. | [Conc.] | |
|---|---|---|---|---|---|
| substrate | vitronectin XF | StemCell Technologies | 7180 | | |
| | CellAdhere Dilution buffer | StemCell Technologies | 7183 | | |
| media | DMEM-F12 | ThermoFisher Scientific - Gibco | 10565-018 | 42% | |
| | Neurobasal | ThermoFisher Scientific - Gibco | 21103-049 | 42% | |
| supplements | KSR | ThermoFisher Scientific - Gibco | 10828-028 | 10% | |
| | N2 supplement (100X) | ThermoFisher Scientific - Gibco | 17502-048 | 0.5% | |
| | B27 supplement (50X) | ThermoFisher Scientific - Gibco | 17504-044 | 1% | |
| | ITS-X supplement (100X) | ThermoFisher Scientific - Gibco | 51500-056 | 2% | |
| | MEM non-essential amino acids | ThermoFisher Scientific - Gibco | 11140-050 | 100 | μM |
| | L-Glutamine | ThermoFisher Scientific - Gibco | 25030-081 | 1 | mM |
| | recombinant human LIF | PeproTech | AF-300-05 | 20 | ng/mL |
| | (100 μg/mL) | | | | |
| small | CHIR99021 | Tocris | 4953 | 3 | μM |
| molecules | | Sigma | SML1046 | | |
| | | biogems | 2520691 | | |
| | | Stemgent | 04-0004 | | |
| | PD0325901 | Sigma | PZ0162 | 2 | μM |
| | | biogems | 3911091 | | |
| | | Stemgent | 04-0006 | | |
| | XAV939 | Sigma | X3004 | 4 | μM |
| | Go6983 | Selleckchem.com | S2911 | 2 | μM |
| | Y-27632 (ROCK inhibitor) | Stemgent | 04-0012 | 1 μM | |
| | | StemRD | Y-001 | (passaging) | |
| | BSA | Sigma | A3311 | 50 | mg/mL |

In certain embodiments, the preparation of 500 mL LIF-4i media can comprise the steps of:
(1) Weighing of 2.5 g BSA and transfer in a bottle.
(2) Gently adding 210 mL DMEM-F12 and 210 mL Neurobasal without making an emulsion with BSA.
(3) Adding 50 mL KSR, 5 mL 100 mM L-Glutamine, 5 mL 100X NEAA, 10 mL ITS-X supplement, 5 mL B27 supplement, 2.5 mL N2 supplements.
(4) Adding 100 μL human LIF (100 μg/mL stock), 15 μL CHIR99021 (100 mM stock in DMSO), 10 μL PD0325901 (100 mM stock in DMSO), 20 μL XAV939 (100 mM stock in DMSO), 20 μL Go6983 (50 mM stock in DMSO).
(5) Warming up the medium at 37° C. until all DMSO and BSA are resuspended and sterile filter.

In certain embodiments, the present disclosure is directed to methods for generating a population of therapeutic progenitor cells under clinical grade, cGMP-compliant conditions, comprising: (a) contacting a population of hiPSCs with a tankyrasc/PARP-inhibitor-containing composition comprising: (i) leukemia inhibitory factor; (ii) a GSK3β signaling pathway inhibitor; (iii) a MEK signaling pathway inhibitor; (iv) a non-specific tankyrase/PARP inhibitor; and (v) a PKC inhibitor; (b) performing FF and XF culture of the hiPSCs to generate a population of therapeutic progenitor cells under clinical grade, cGMP-compliant conditions. In certain embodiments, the tankyrase/PARP inhibitor is selected from: XAV939, IWR-1, G007-LK, JW55, AZ1366, JW 74, NVP-TNKS656 and combinations thereof. In certain embodiments, the GSK3β signaling pathway inhibitor is selected from: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M), N-(4-methoxybenzyl)-N′-(5-nitro-1,3-thiazol-2-yl)urea (AR-A014418) and combinations thereof. In certain embodiments, the MEK signaling pathway inhibitor is selected from: PD032590, CI-1040 (PD184352), cobimetinib (GDC-0973, XL518), Selumctinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 and combinations thereof. In certain embodiments, the PKC inhibitor is Go6983.

In certain embodiments, the present disclosure is directed to methods for generating a population of therapeutic progenitor cells under clinical grade, cGMP-compliant conditions, wherein the hiPSCs used to generate the therapeutic progenitor cells are derived from primed hiPSCs. In certain embodiments, the priming of hiPSCs comprises contacting the hiPSCs with a serum replacer-containing media. In certain embodiments, the priming hiPSCs comprises contacting the hiPSCs with a ROCK inhibitor. In certain embodiments, the ROCK inhibitor is Y-27632.

In certain embodiments, the present disclosure is directed to methods for generating a population of therapeutic progenitor cells under clinical grade, cGMP-compliant conditions, wherein the population of therapeutic progenitor cells are generated from a population of HLA-defined hiPSCs. In certain embodiments, the population of HLA-defined hiPSCs comprise human CD34+ cord blood cells.

In certain embodiments, the present disclosure is directed to methods for generating a population of therapeutic progenitor cells under clinical grade, cGMP-compliant conditions comprising a FF and XF culture of the hiPSCs to generate a population of therapeutic progenitor cells under clinical grade, cGMP-compliant conditions, wherein the FF and XF culture of the hiPSCs in contact with the tankyrase/PARP inhibitor-continuing composition is comprises culture of the hiPSCs on vitronectin-coated substrates. In certain embodiments, the FF and XF culture of the hiPSCs in contact with the tankyrase/PARP inhibitor-containing composition for 1 to 10 passages. In certain embodiments, passages 1-3 of the FF and XF culture of the hiPSCs in contact with the tankyrase/PARP inhibitor-containing composition comprise selection of dome-shaped colonies for each subsequent passage.

In certain embodiments, the modified LIF-4i method provides a completely defined XF/FF culture system for the derivation and long-term cryopreservation and storage (TABLE 2) of clinical-grade TIRN-hiPSCs, e.g., from HLA-typed CB or PBSC donors.

TABLE 2
Cryopreservation and Storage

| | LIF-4i crypreservation medium |
|---|---|
| | (10 mL) |
| LIF-4i medium (Table 1) | 50% |
| w/o PD0325901 and Go6983 | |
| KnockOut serum replacement | 40% |
| dimethyl sulfoxide (DMSO) | 10% |
| Y-27632 | 5 μM |

### 5.3 Production of cGMP-Grade Banks of “Universal” UTIRN-hiPSCs

In another aspect, the present disclosure relates to the production of cGMP-grade banks of clinical grade “universal” UTIRN-hiPSCs. UTIRN-hiPSC reversion can be used to revert either established or commercially available transgene-free FF hiPSC. With this method, established conventional, primed hESC/hiPSC (or CB-hiPSC derived entirely with FF/XF episomal CB reprogramming) that are cultured in FF E8 medium can be rapidly reverted in bulk with LIF-4i to an FF naïve-like state. FF TIRN-hiPSC or UTIRN-hiPSC are cultured on vitronectin-coated vessels in physiological (5%) O₂, and can be cryopreserved, or used directly for differentiation without further manipulation. In addition to enhanced functionality, UTIRN-hPSC possess multiple advantages over conventional hPSC culture, and over other LIF-3i/MEF naïve reversion systems. These advantages include, in certain embodiments, ease of single cell passaging in a defined, reproducible FF system that uses pre-screened, high-quality commercially-available components. As outlined herein, episomally-reprogrammed FF/XF cGMP-compliant HLA-defined CB-derived UTIRN-hiPSC lines from start to finish have been established. The same methods can be employed with clinical grade CD34+ PBSC or BM, among other cell types.

Multiple other culture systems have also been reported to promote conventional hPSC to similar naïve-like pluripotent states. Although these hPSC culture systems have also relied on utilization of classical mouse naïve 2i conditions, in most cases these single-cell passaging methods also required additional chemical modulation for stabilizing an inherently unstable/metastable human naïve state. Unlike the TIRN system, most of these other methods demonstrated impaired functional pluripotency following differentiation and/or acquired abnormal epigenomic imprints or karyotypes. Although the emergence of abnormal karyotypes within conventional primed hPSC cultures is already well documented, prolonged, enzymatic single-cell passaging methods that are routinely employed in most naïve reversion methods, have also been shown to potentiate the generation of abnormal chromosomal configurations; more sensitive techniques (e.g., copy number variations, single nucleotide polymorphism) may reveal additional alterations.

In contrast, UTIRN-hPSC lines not only possessed normal karyotypes at low-medium passages (e.g., p5-p15), as well as at high passage numbers (e.g., >p30) following LIF-4i FF TIRN culture, but also improved genomic stability (as assessed by DSB DNA repair studies). Using the sensitive allele-specific Infinium methylation array platform, it was also previously demonstrated that CpG methylation marks at imprinted loci of a wide repertoire of LIF-3i/MEF-reverted hPSC lines (following 4-7 passages in LIF-3i) were found to be grossly normal in structure. Since abnormal genomic imprints and karyotypes may ultimately impair functional capacity of hPSC, prerequisite guidelines were outlined in this protocol that encourage researchers to validate hPSC cultures before and after naïve reversion, using this method as well as others.

In certain embodiments, the LIF-4i FF TIRN methods described herein can improve functional pluripotency across germ layers in a large repertoire of hESC and non-transgenic hiPSC lines. Unlike other naïve reversion protocols, the methods described herein do not require a re-priming step for subsequent differentiation of TIRN-hPSC (i.e., converting TIRN-hPSC back to conventional primed conditions prior to their use in directed differentiation assays). In certain embodiments, the LIF-4i FF-reverted TIRN-hPSC displayed significantly more efficient differentiation capacities than their isogenic conventional hPSC counterparts in both teratoma assays and directed differentiation protocols of lineages of all three germ layers. Due to assay-dependent and interline variations in functional testing of conventional hPSC, lineage-specific differentiation should be evaluated using independent directed differentiation protocols and hPSC derived from multiple genetic backgrounds. Using careful experimental design, a broad array of hPSC lines can be expected to significantly improve their multi-lineage differentiation efficiencies compared to their isogenic conventional counterparts following 4-10 passages in LIF-4i TIRN conditions.

For example, but not by way of limitation, the methods and compositions described herein have been used to generate and functionally validate pre-clinical naïve vascular progenitors (N-VP) from a test bank of representative UTIRN-hiPSC lines derived in cGMP-compliant conditions from HLA-defined CD34+ CB as a pilot for future Phase I/II clinical trials.

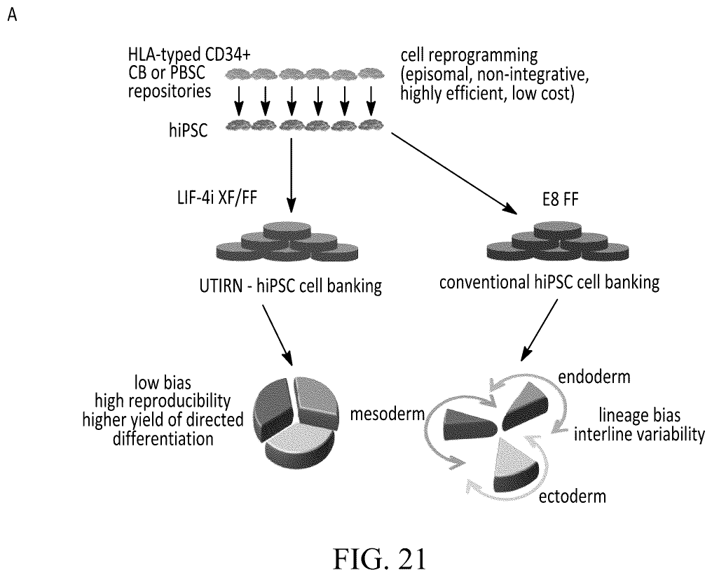

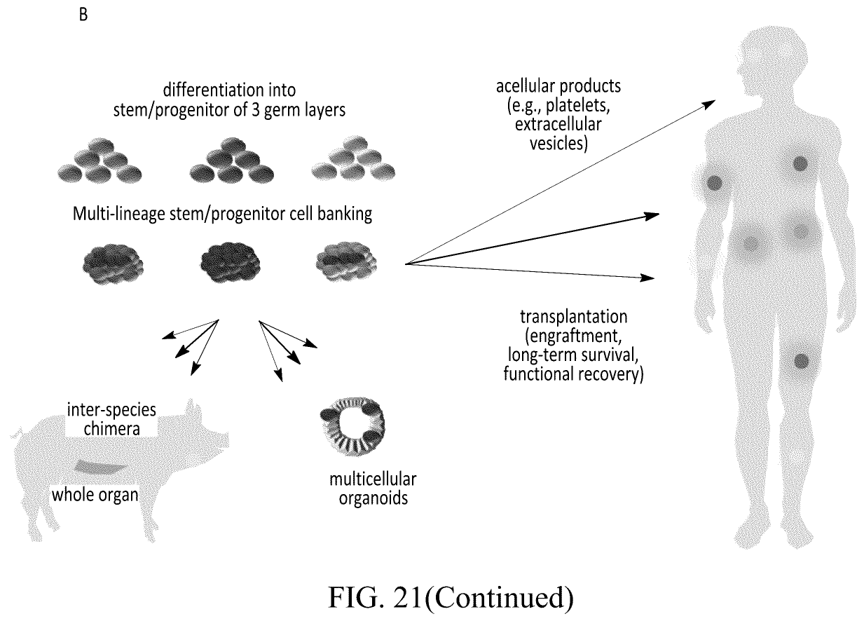

In summary, the TIRN reversion method rapidly and clonally expands the numbers of hPSC, improves their downstream differentiation efficiency, increases lineage-committed progenitor cell numbers following differentiation, and decreases interline variability of conventional, lineage-primed hPSC lines (FIG. 21A). UTIRN-hPSC with improved functionality can also have wide impact as contributing functional tissues to a developing embryo. For example, stable UTIRN-hESC could be employed for developing transplantable human organs and adult stem cells in developing interspecies human-animal chimeras, or for generating humanized gene-targeted animal models of disease (FIG. 21B).

### 6. EXEMPLARY EMBODIMENTS

In certain embodiments, the presently disclosed subject matter provides methods for generating a population of therapeutic progenitor cells under clinical grade, current good manufacturing practice (cGMP)-compliant conditions, comprising contacting a population of human induced pluripotent stem cells (hiPSCs) with a tankyrase/PARP-inhibitor-containing composition. In certain embodiments, the tankyrasc/PARP-inhibitor-containing composition comprises (i) leukemia inhibitory factor (LIF), (ii) a Glycogen Synthase Kinase 3-β (GSK3β) signaling pathway inhibitor, (iii) a mitogen-activated protein kinase (MEK) signaling pathway inhibitor, (iv) a tankyrasc/PARP inhibitor, and (v) a protein kinase C (PKC) inhibitor. In certain embodiments, the method further comprises performing feeder-free (FF) and xeno-free (XF) culture of the hiPSCs to generate a population of these therapeutic progenitor cells under clinical grade, cGMP-compliant conditions.

In certain embodiments, the tankyrase/PARP inhibitor is selected from: XAV939, IWR-1, G007-LK, JW55, AZ1366, JW 74, NVP-TNKS656 and combinations thereof. In certain embodiments, the GSK3β signaling pathway inhibitor is selected from: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M), N-(4-methoxybenzyl)-N′-(5-nitro-1,3-thiazol-2-yl)urea (AR-A014418) and combinations thereof. In certain embodiments, the MEK signaling pathway inhibitor is selected from: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 and combinations thereof. In certain embodiments, the PKC inhibitor is Go6983.

In certain embodiments, the hiPSCs are derived from primed hiPSCs. In certain embodiments, the priming of hiPSCs comprises contacting the hiPSCs with a serum replacer-containing media. In certain embodiments, priming the hiPSCs comprises contacting the hiPSCs with a ROCK inhibitor. In certain embodiments, the ROCK inhibitor is Y-27632.

In certain embodiments, the population of therapeutic progenitor cells are generated from a population of HLA-defined hiPSCs. In certain embodiments, the population of HLA-defined hiPSCs comprise human CD34+ cord blood cells.

In certain embodiments, the FF and XF culture of the hiPSCs in contact with the tankyrase/PARP inhibitor-continuing composition comprises culturing the hiPSCs on vitronectin-coated substrates. In certain embodiments, the FF and XF culture of the hiPSCs in contact with the tankyrase/PARP inhibitor-containing composition is performed for 1to 10 passages. In certain embodiments, passages 1-3 of the FF and XF culture of the hiPSCs in contact with the tankyrase/PARP inhibitor-containing composition comprise selection of dome-shaped colonies for each subsequent passage.

### 7. EXAMPLES

The following examples are merely illustrative of the presently disclosed subject matter and should not be considered as limitations in any way.

### Example 1: Derivation of Clinical-Grade, Xeno-Free (XF), Feeder-Free (FF) UTIRN-hiPSC with the LIF-4i Chemical Method

The instant disclosure describes strategies to efficiently revert feeder-free (FF), lineage-primed, conventional human pluripotent stem cells (hPSC) into a naïve epiblast-like pluripotent state with improved functionality, and to maintain this novel stem cell state in FF, xeno-free (XF), clinical-grade culture conditions for downstream cell therapeutic applications.

Previously disclosed feeder-dependent LIF-3i/MEF culture systems are not stable in the absence of feeders and TIRN-hiPSC undergo spontaneous differentiation. Thus, a two-step LIF-4i XF/FF TIRN method (TABLE 1) was developed following extensive media analysis of the LIF-3i/MEF protocol.

The LIF-4i FF chemical TIRN reversion method employs the classical leukemia inhibitory factor (LIF), GSK3β, and MEK/ERK inhibition cocktail (LIF-2i), supplemented with only the non-specific tankyrase/PARP inhibitor XAV939 in a novel completely FF protocol. To stabilize TIRN-hPSC without feeders, the substrate and media composition were modified from the original LIF-3i/MEF method, and a 4th inhibitor (i.e., Go6983) targeting protein kinase C (LIF-4i FF medium) was introduced, which had been shown to reinforce pluripotency in human primed and rodent ESC. This mechanism is independent from STAT3 activation and MEK/GSK3β inhibition and antagonizes lineage commitment. This inhibitor has previously been introduced into other human naïve culture systems to minimize spontaneous differentiation.

The modified LIF-4i TIRN method provides a completely defined XF/FF culture system for the derivation and long-term cryopreservation and storage (TABLE 2) of clinical-grade UTIRN-hiPSC from HLA-typed CB or PBSC donors.

Like the previously described feeder-dependent LIF-3i/MEF method, LIF-4i FF conditions revert conventional hPSC to a stable pluripotent state adopting biochemical, transcriptional, and epigenetic features of the human pre-implantation epiblast. The LIF-4i FF method, however, requires minimal cell culture manipulation and is highly reproducible in a broad repertoire of human embryonic stem cell (hESC) and transgene-free human induced pluripotent stem cell (hiPSC) lines. Moreover, this LIF-4i FF TIRN reversion method does not require a ‘re-priming’ step prior to differentiation; TIRN-hPSC can be differentiated directly with extremely high efficiencies and maintain karyotypic and epigenomic stabilities (including at imprinted loci).

To broaden the utility of the method, the LIF-4i FF TIRN protocol described herein was evaluated to promote universal naïve reversion with almost any conventional hESC or transgene-free hiPSC line cultured in standard feeder-free hPSC conditions (e.g. E8). This universalized naïve reversion method employs continuous culture on a single substrate (i.e., vitronectin), as well as selection of cell densities and bulk passaging of conventional hPSC without any requirement for tedious colony picking. Dissociation methods were selected to promote bulk enrichment of emerging naïve hPSC during the initial passages, so that adapted naïve hPSC lines could subsequently tolerate bulk clonal passaging of naïve-reverted cells in LIF-4i FF TIRN conditions. The methods described herein obviate the need for picking and subcloning of rare ‘stable’ colonies and promote rapid expansion of naïve epiblast-like hPSC.

The LIF-4i FF TIRN reversion methods described herein markedly improve the functional pluripotency of a broad repertoire of conventional hPSC by decreasing their lineage-primed gene expression and erases the interline variability of directed differentiation commonly observed amongst independent primed, conventional hPSC lines

The LIF-4i FF TIRN methods described herein have been successfully used to stably expand and maintain a broad repertoire of >10 independent, genetically diverse conventional hPSC lines for >10-30 passages using either non-enzymatic or enzymatic dissociation methods, and without evidence of induction of chromosomal or epigenomic abnormalities, including at imprinted loci.

The LIF-4i FF TIRN methods described herein have great utility in regenerative medicine and cellular therapies, and can be utilized to expand UTIRN-hiPSC derived from HLA-defined CD34+ CB or PBSC donors. Representative characterizations of LIF-4i FF-reverted TIRN-hPSC are provided, and experimental strategies for functional comparisons of isogenic hPSC in lineage-primed vs. naïve-like states are outlined herein.

### 1.1 Xenofree (XF) Feeder-Free (FF) Reprogramming of HLA-Defined CB Cells into XF/FF UTIRN-hiPSC

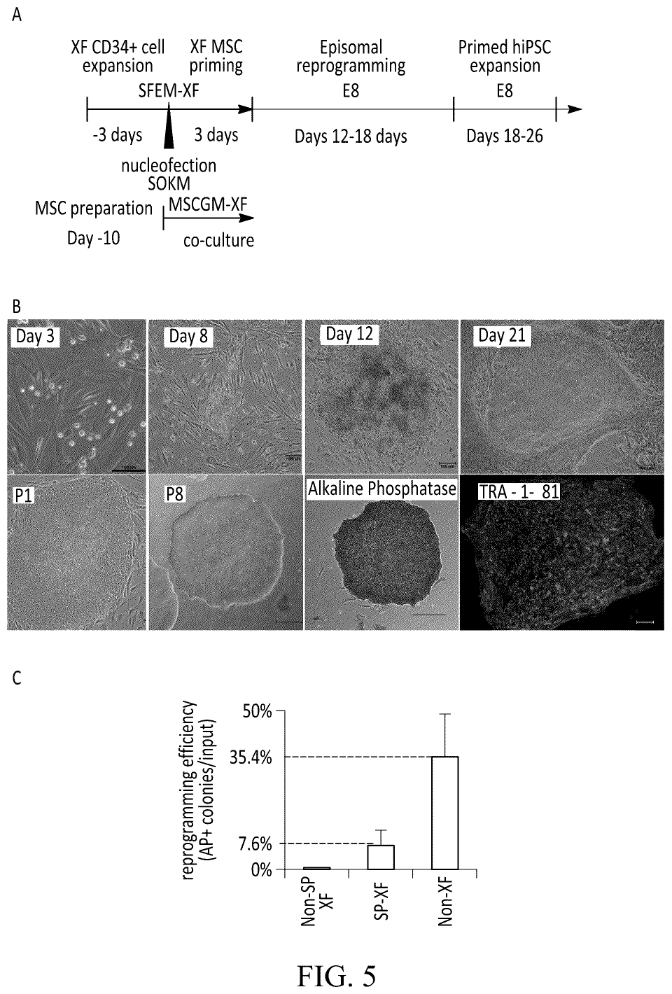

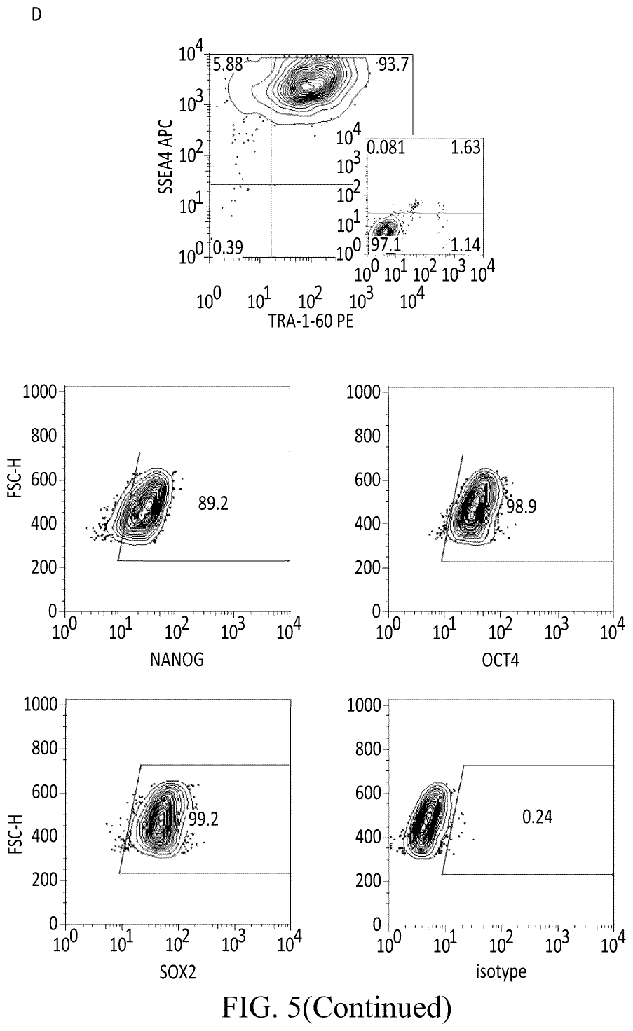

The schematic, images, and reagents of the XF/FF episomal reprogramming of human CD34+ cord blood (CB) cells of pre-defined HLA type are summarized in FIG. 5A, 5B and TABLE 3.

TABLE 3

| | XF condition |
|---|---|
| MSC medium | XF MSC medium (Life Technologies, |
| | A10675-01) |
| Pluripotency expansion | E8 medium (including 100 ng/mL |
| medium | bFGF & 1.5 ug/mL TGFb) |
| Substrate | Human vitronectin |
| Feeder | none |
| Enzyme | Versene |
| Freezing solution | 50% XF Serum replacement |
| | 40% E8 medium |
| | 10% DMSO |
| | 5 uM Rock inhibitor |
| Washing solution | 90% E8 medium |
| for thawing | 10% XF Serum replacement |

Episomal FF/XF reprogramming of CD34+ CB, PBSC, or BM methods can be performed in a cGMP-compliant environment with cGMP-compliant reagents from start to finish. For example, to derive HLA-defined UTIRN-hiPSC, GTP-grade HLA-defined CB, PBSC, or BM can first be obtained from a cryopreserved bank or commercial source, e.g., following prospective donor consent of HLA screened donors, from an approved IRB protocol and according to institutional requirements. In certain instances, selection of O-negative HLA-homozygous donors is associated with additional advantages.

CD34+-enriched cord blood (CB) cells from mixed donors were commercially obtained (AllCells), and thawed in cGMP-grade XF StemSpan Serum-Free Expansion Medium (SFEM)-XF medium (StemCell Technologies) supplemented with 50 ng/ml human recombinant FLT3 ligand, 10 ng/mL human recombinant thrombopoietin (TPO), and 50 ng/mL human recombinant Kit-ligand (FTK), and cultured for 3 days onto ultra-low attachment surfaces (Corning). Expanded CD34+ progenitors were nucleofected (AMAXA Nucleofector II, Lonza) with 6 ug per million of cells of a single EBNA-based episomal vector that expressing sequences for SOX2, OCT4, KLF4, and c-MYC.

Nucleofected cells were plated (17,500 cells per cm²) over irradiated human mesenchymal stem cells (MSC) onto a XF synthetic peptide substrate (Corning Synthemax) in SFEM-XF medium supplemented with human recombinant FTK and 10μM ROCK inhibitor Y-27632. An equal volume of XF E8 medium was added 2 days later and half of the medium was replaced with fresh E8 medium on the third day (FIG. 5B). Subsequently, half of the medium was changed every other day. Floating cells were recovered during initial medium changes and added back to the culture. After 18 to 21days (FIG. 5B), XF-hiPSC colonies arised and were manually picked using an inverted microscope within a biosafety cabinet and transferred onto plates coated with Vitronectin-XF peptide (StemCell Technologies) in E8 medium supplemented with 5 μM ROCK inhibitor. The next day, the medium was replaced with E8 medium without ROCK inhibitor supplementation. XF-hiPSC were continuously expanded in E8 medium thereafter with daily changes and passaged using EDTA-based Versene cell dissociation solution (Gibco, ThermoFisher) (FIG. 5B). Primed, conventional CB-hiPSC expanded in E8 and expressing surface pluripotency markers were readily generated at efficiencies approaching 8% using this XF/FF method (FIG. 5C, 5D).

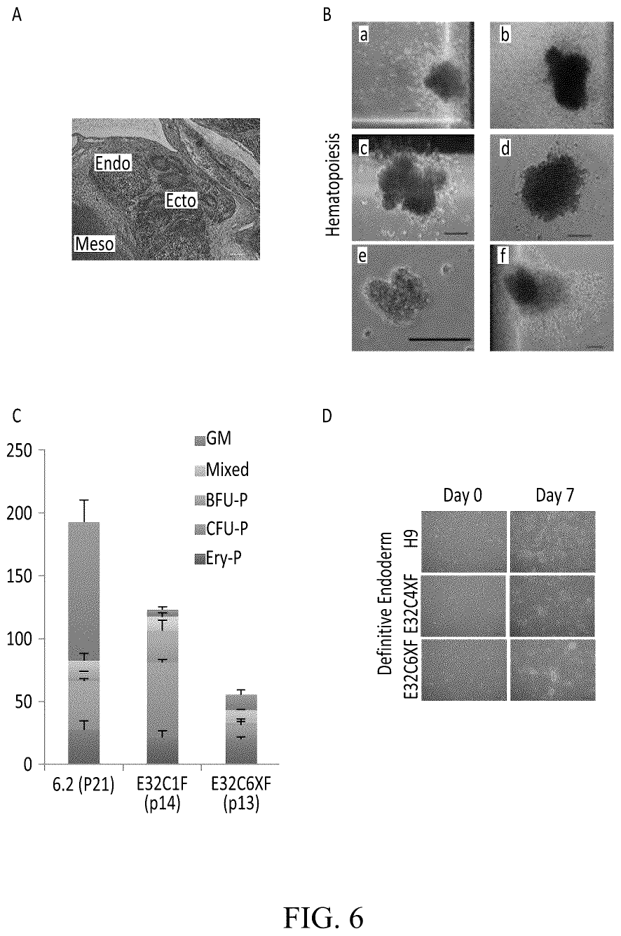

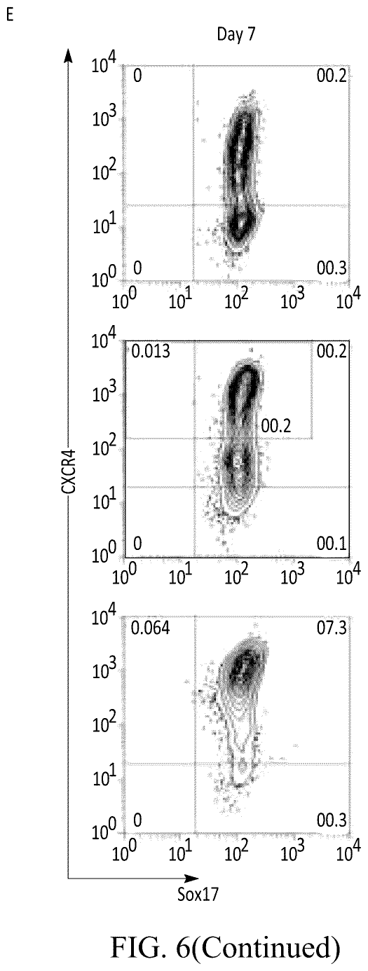

Stable, conventional, primed XF/FF UTIRN-hiPSC colonies arising after 10-20 days were passaged non-enzymatically and cryopreserved with 50% XF SR/40% E8/10% DMSO prior to LIF-4i naïve reversion onto vitronectin-XF-coated plates. XF/FF cGMP-compliant UTIRN-iPSC lines need to be quality control characterized for TRA+ markers, karyotypes, SNP genomic arrays, mycoplasma/endotoxin assay, tri-lineage teratoma assay, and Southern blotting/PCR studies for verification of lack of vector integration. These primed XF/FF CB-hiPSC were validated for multi-lineage functional pluripotency by teratoma assays (FIG. 6A) and in vitro hematopoietic and endodermal directed differentiation assays (FIG. 6B-6E, TABLE 4).

TABLE 4
Monolayer Defined Differentiation (XF Condition)

| | Monolayer Defined |
|---|---|
| | Differentiation |
| | (XF condition) |
| Mesodermal | APEL including |
| determination | CHIR9902 |
| | Activin A |
| | BMP4 |
| | VEGF |
| Vascular | APEL including |
| differentiation | SB431542 (10 uM) |
| | VEGF (50 ug/mL) |
| Vascular endothelial cell | Modified EGM2, Lonza |
| expansion | (hEGF |
| | hydrocortisone |
| | 5% XF serum replacement |
| | VEGF |
| | hFGF-B |
| | IGF-1 |
| | ascorbic acid |
| | heparin) |

### 1.2 Adaptation of Conventional, Primed Feeder-Free (FF) HLA-Defined CB-hiPSC to Stable Naïve Reversion in FF/XF LIF-4i

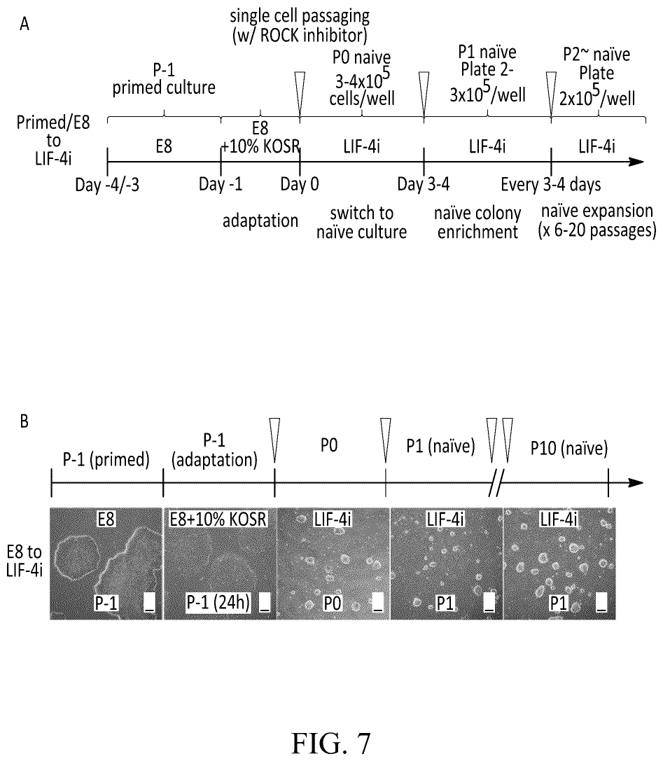

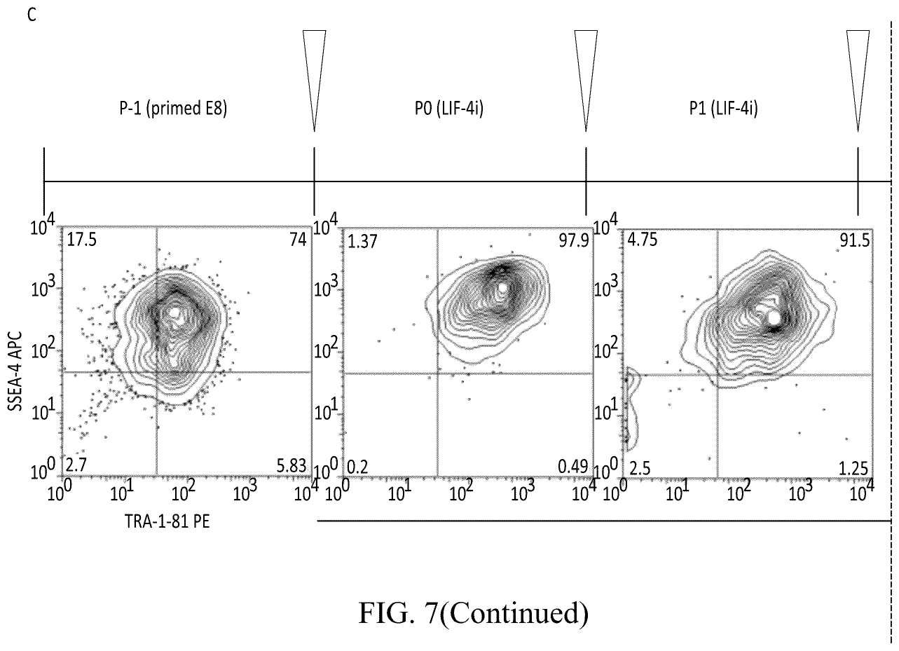

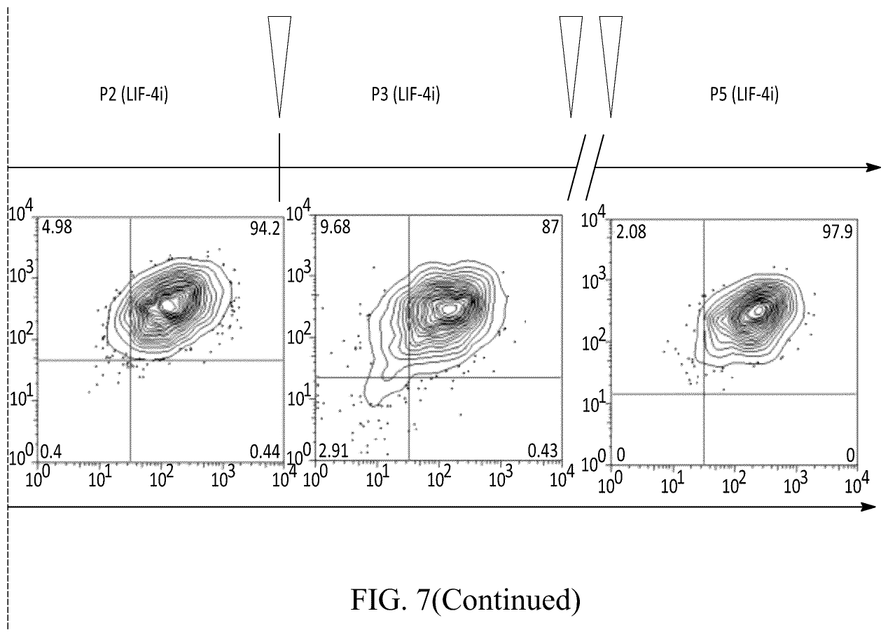

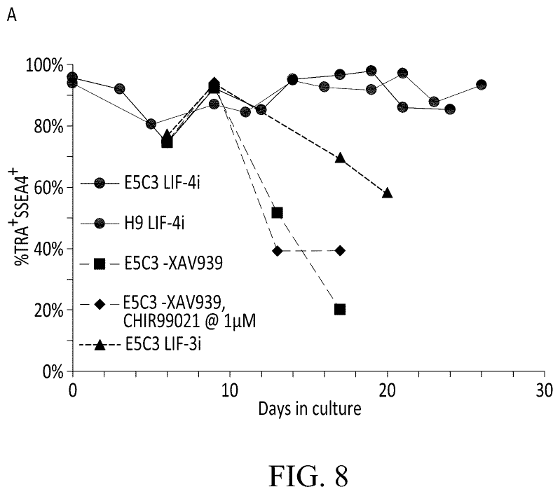

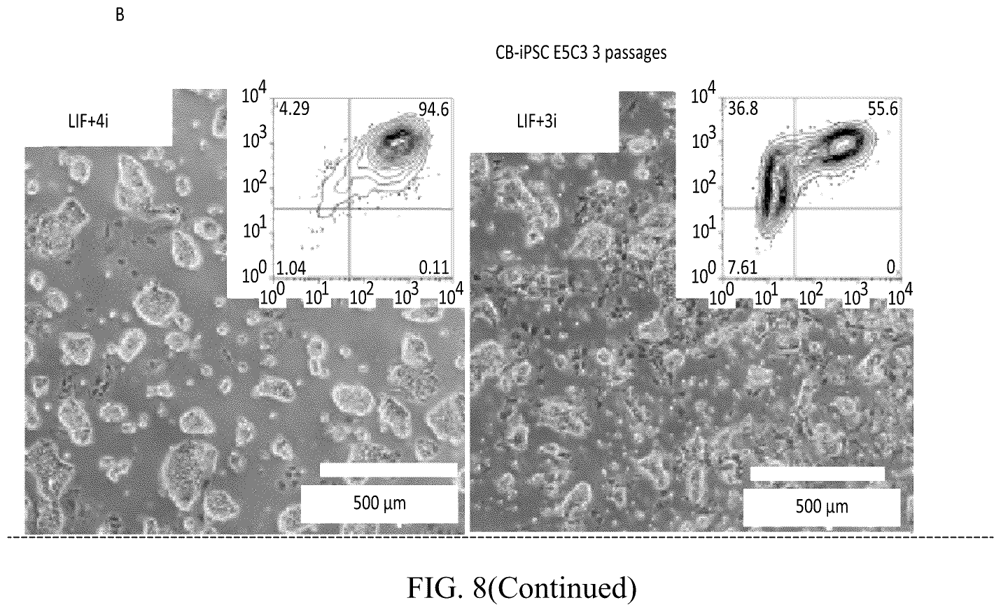

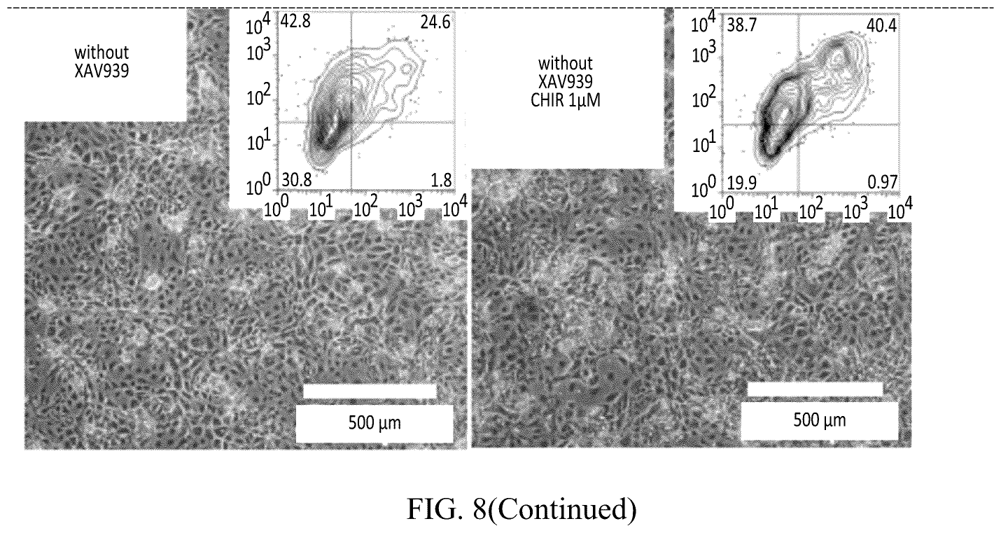

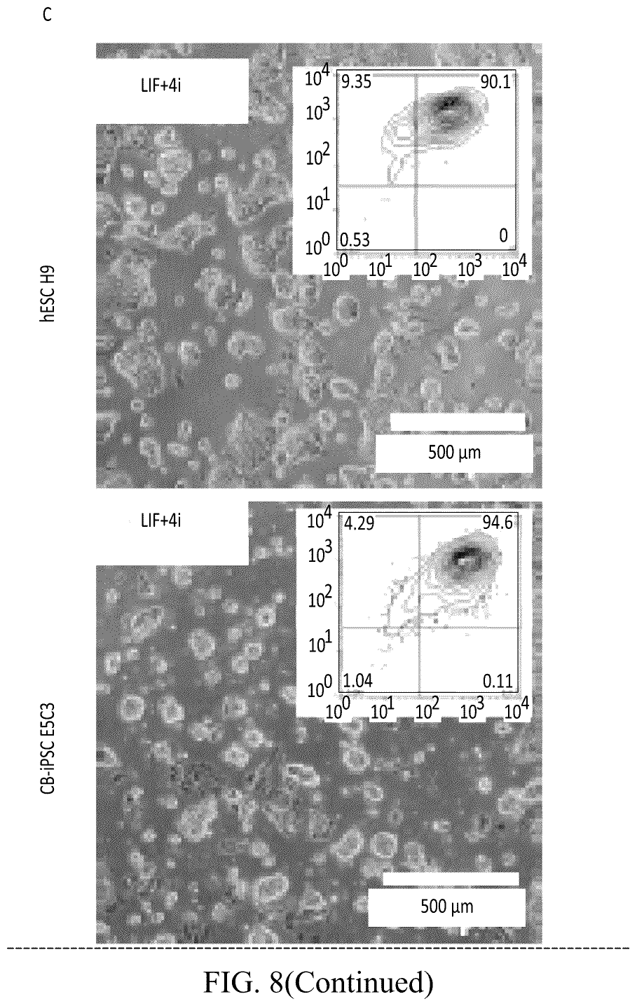

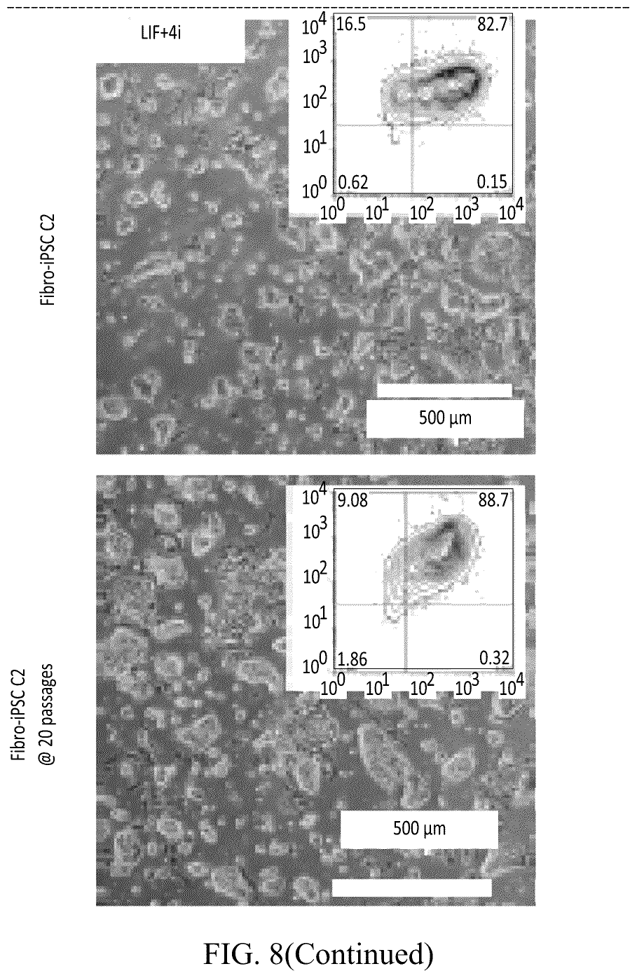

The schematic, images, and reagents of the LIF-4i XF/FF TIRN reversion method of primed, conventional XF/FF hiPSC cells of a pre-defined HLA type to XF/FF UTIRN-hiPSC are summarized in FIG. 7, FIG. 8, and TABLE 1.

Before beginning, all conventional, primed hPSC lines growing in FF conditions can be verified to possess a normal human karyotype by G-banding, prior to beginning TIRN reversion. LIF-4i FF TIRN reversion of high-passage conventional hiPSC lines (e.g., P>40-50) should be avoided, as such cultures may already harbor genomic aberrations that can negatively impact stable, efficient, and bulk LIF-4i FF reversion of primed hiPSC. In general, an effort should be made to revert conventional hPSC lines at the lowest possible passage that they are available.

LIF-4i XF/FF TIRN reversion is compatible with conventional hPSC starting from either feeder-dependent or feeder-free primed culture conditions, but feeder-free primed culture systems (e.g., E8, mTSER) are often desirable for commercial production purposes. Non-enzymatic methods (i.e., EDTA-based dissociation buffers) are also useful for passaging of conventional hPSC prior to preparing them for reversion. LIF-4i formulation (TABLE 1) does not contain antibiotic or antifungal agents. Thus, standard operation rules for biosafety cabinet sterility and maintenance can be observed to avoid any bacterial or fungal contamination.

Before switching to LIF-4i XF/FF TIRN conditions, primed hiPSC can be passaged in a biosafety cabinet (standard passaging in E8 on vitronectin XF is recommended, but not required) and allowed to reach ˜30% confluency (i.e., 3-4 days after initial plating) in a CO₂ incubator (5% CO₂, humid atmosphere).

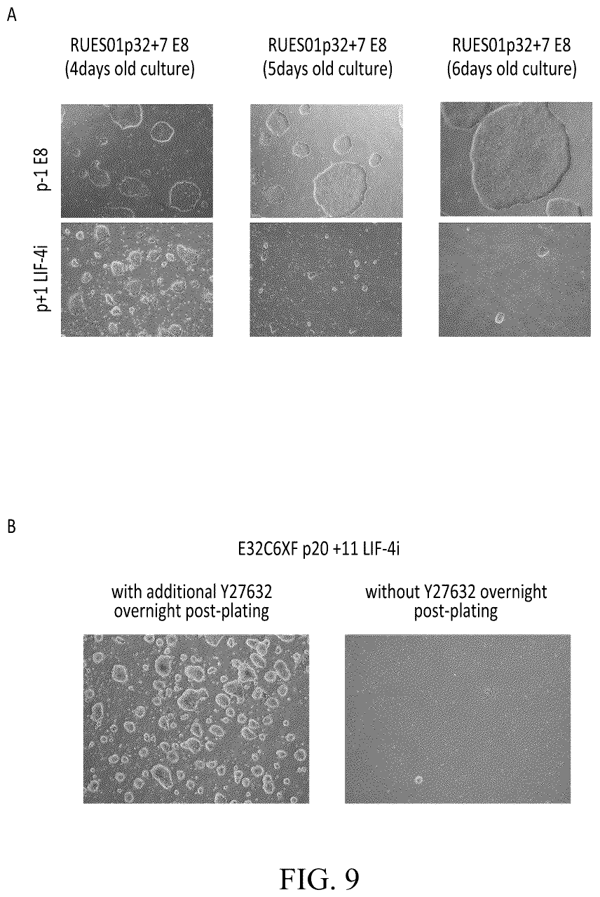

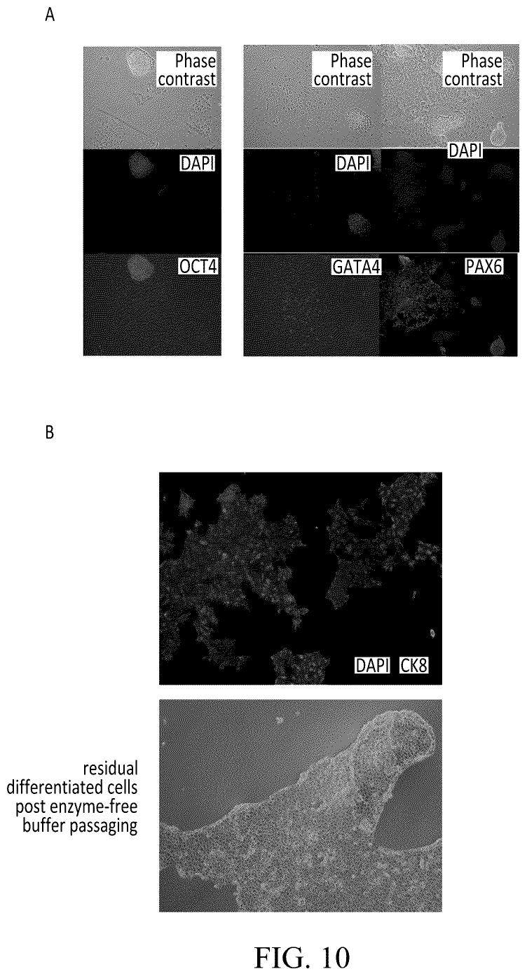

Primed hPSC cultures that are over-expanded prior to switching to LIF-4i (i.e., >50% confluency) can display pronounced differentiation (i.e., primarily extraembryonic lineages such as cytokeratin 8+ primitive endoderm) for up to 3 passages after transition in LIF-4i (FIG. 9, FIG. 10). These cultures may then require extensive colony manual picking during the first passages in LIF-4i. The following protocol has been developed to avoid such strenuous and highly selective passaging steps.

Primed hPSC cultures that were expanded using defined, serum-free medium that did not include any Knock-out Serum Replacer (e.g., E8 medium), can be supplemented with 10% Knock-out Serum Replacer in a biosafety cabinet and incubated for at least one hour (up to overnight incubation) in a CO₂ incubator (5% CO₂, humid atmosphere) to adapt them prior to their subsequent passage and stable reversion in LIF-4i (FIG. 9A). Direct transition to LIF-4i from conventional media that do not contain any amount of Knock-out Serum Replacer will typically result in significantly reduced clonal efficiencies during the first passage in LIF-4i (FIG. 9A).

Prior to passaging, conventional hPSC culture plates are placed in a biosafety cabinet, supernatant is discarded and cells are washed once with 2 mL PBS. PBS wash can be gently aspirated and 1 mL of non-enzymatic EDTA-based cell dissociation buffer (e.g., enzyme-free Gibco cell dissociation buffer) can be added to each well. The cell can then incubate for 5 min at 37° C. in a CO₂ incubator.

The cells can then be gently triturated with a 1 mL pipette to obtain a single cell suspension in a biosafety cabinet. The cell suspension can then be collected in LIF-4i medium (Table 3, at least 2-fold dilution) in sterile 15 ml conical tubes.

The cell suspension collected in LIF-4i medium can then be centrifuged at 200 g for 5 min, the supernatant aspirated/discarded, and the cell pellet resuspended in 1 mL of LIF-4i medium in a biosafety cabinet for cell counting using a hemocytometer or an automatic cell counter.

3×10⁵ cells can be distributed into 2 mL of LIF-4i medium supplemented with 5 μM Y-27632 onto 1 well of Vitronectin-XF-coated plate. Initial plating efficiency will vary between hPSC cultures and may need to be individually assessed. The plate can then be placed in a CO₂ incubator (5% O₂, 5% CO₂, humid atmosphere).

Stable SSEA4+TRA-1-81 LIF-4i FF UTIRN-hiPSC cultures (FIG. 7C) can generally be more efficiently maintained using physiologic (5% O₂) oxygen levels. The maintenance of fresh stocks of small molecules (TABLE 1) is often desirable, especially stocks of the tankyrase/PARP inhibitor XAV939, which is useful for maintaining a naïve pluripotent state during long term culture (FIG. 8). The supplementation of LIF-4i with an anti-apoptotic chemical (i.e. 5 μM Y-27632) can be used for overnight incubation post-plating to allow efficient cell attachment and clonal cell survival of UTIRN-hiPSC (FIG. 9C).

The next day, the plate can be gently swirled to lift all non-attached cells, the medium can be aspirated and replaced with 2 mL of LIF-4i medium in a biosafety cabinet daily for 2-3 days or until cells are 60-70% confluent. The plate can be placed in a CO₂incubator (5% O₂, 5% CO₂, humid atmosphere).

### 1.3 Long-Term Maintenance and Expansion of UTIRN-hiPSC in LIF-4i FF Medium

Following initial adaptation to LIF-4i, subsequent LIF-4i TIRN cultures are passaged every 2-3 days, or when cultures become 60-70% confluent in a biosafety cabinet (FIG. 7A, 7B).

Typically, LIF-4i XF/FF TIRN cultures require rigorous maintenance and allowing UTIRN-hPSC cultures to reach high confluency/cell density from prolonged culture (e.g., >4 days) decreases subsequent clonal re-plating efficiency, and promotes spontaneous differentiation (FIG. 10).

While LIF-4i XF/FF TIRN reversion of high-passage (e.g., >p40) lineage-primed, conventional hPSC lines is possible, generally an effort should be made to revert conventional hPSC lines at the lowest possible passage that they are available. Additionally, the use of LIF-4i-reverted hPSC that have undergone greater than 15 LIF-4i passages is not recommended for functional studies, since such TIRN-hPSC cultures may harbor karyotypically-abnormal clones due to prolonged clonal cell culture selection. Fresh LIF-4i reversions of low-passage conventional hPSC lines should be conducted for functional studies, if stocks of TIRN-hPSC with <10 passages in LIF-4i are not available.

The small molecule Go6983 is auto-fluorescent. Cells may be recovered and resuspended for counting using LIF-4i medium that does not include Go6983 before passaging in complete LIF-4i FF medium when fluorescence-based assays are required. Multiple washes in medium without Go6983 and PBS are recommended to permit cellular exclusion of Go6983 and minimize carry-over of red auto-fluorescence.

UTIRN-hiPSC are typically passaged for least 4-7 continuous bulk passages in LIF-4i FF TIRN medium prior to use of UTIRN-hiPSC in functional studies or cryopreservation. In general, it is desirable to record the number of passages of TIRN-hPSC in either conventional or LIF-3i media.

The use of LIF-4i-reverted UTIRN-hiPSC that have undergone greater than 10 LIF-4i passages is not recommended for functional studies, since such TIRN-hiPSC cultures might harbor karyotypically-abnormal clones due to prolonged clonal cell culture selection. Fresh LIF-4i reversions of low-passage conventional hiPSC lines should preferably be conducted for functional studies, if stocks of UTIRN-hPSC with <10passages in LIF-3i are not available.

The initial LIF-4i FF passage will often only display minimal differentiation (FIG. 7B), but two types of colonies may arise from the original single cell plating: (1) small dome-shape colonies and (2) flatter primed-like colonies. Smaller dome-shape colonies may be manually picked at this step for further expansion, but the protocol steps indicated below can also be used to achieve bulk passaging.

In a biosafety cabinet, the culture medium can be discarded and each well washed of initial LIF-4i passage cultures by gently adding 2 mL of PBS. PBS can be discarded and 1 mL of non-enzymatic EDTA-based dissociation buffer added. This can then be incubated for 5 min at 37° C. in a CO₂ incubator (5% CO₂, humid atmosphere).

Cold PBS for washing cells as TIRN-hPSC is generally disfavored as it may detach from the plate. In addition, extended exposure to PBS or leaving the plate at room temperature are also disfavored, as these conditions may also result in some colony detachment.

Using an inverted microscope or other appropriate means, it can be useful to verify that most dome-shape colonies have detached and ensure the cell suspension is collected, e.g., in a biosafety cabinet. If flat colonies are present, exposure to detachment solution should be limited, so that carry-over of differentiated cells is minimized during the next passages. LIF-4i FF TIRN medium can be added (TABLE 1, at least 2-fold dilution) and the cells gently triturated by pipetting to recover all hPSC in a single cell suspension. The cells can then be transferred, e.g., in sterile 15 ml conical tubes. If a large number of detached cells are still detected in the well under microscope, 1 mL of LIF-4i medium can be added and combined with the TIRN-hPSC in the 15 ml conical.

The 15 ml conical can be centrifuged at 200 g for 5 min and the supernatant aspirated/discarded. The cells can be re-suspended in LIF-4i medium in a biosafety cabinet for cell counting using a hemocytometer or an automatic cell counter.

˜2×10⁵ cells can be plated into 2 mL of LIF-4i FF medium supplemented with 5 μM Y-27632 per well onto Vitronectin-XF-coated 6-well plates in a biosafety cabinet. The plate can be placed in a CO₂ incubator (5% O₂, 5% CO₂, humid atmosphere).

The next day, the plate can be gently swirled to lift all non-attached cells, the medium aspirated and replaced with 2 mL of LIF-4i medium in a biosafety cabinet daily for 2-3 days. The plate can then be placed in a CO₂ incubator (5% O₂, 5% CO₂, humid atmosphere).

The 2nd and 3rd passages in LIF-4i FF require rigorous maintenance (FIG. 7B). Allowing prolonged culture of TIRN-hPSC LIF-4i cultures (e.g., >3 days) during the first 3 passages decreases subsequent clonal efficiency and promotes spontaneous differentiation. The 2nd and 3rd LIF-4i passages may expand juxtaposed monolayer differentiation areas (e.g., cytokeratin 8+ primitive endoderm) and independent dome-shape TIRN-hPSC that may be manually picked for further expansion.

In a biosafety cabinet, culture medium can be discarded and each well of 2^{nd} or 3^{rd} LIF-4i passage cultures washed by gently adding 2 mL of PBS. The PBS can be discarded, and 1 mL of non-enzymatic EDTA-based cell detachment solution can be added. The cells can then be incubated for 5 min at 37° C. in a CO₂ incubator (5% CO₂, humid atmosphere).

Using an inverted microscope or other appropriate means, it can be advantageous to verify that most dome-shape colonies have detached. The sides of the plate can be gently tapped to maximize colony detachment.

Without trituring cells in the well, the cell suspension can be gently transferred in sterile 15 mL conical tubes in a biosafety cabinet. 1 mL of LIF-4i medium can be added (TABLE 1; at least 2-fold final dilution) without disturbing the monolayer areas of differentiated cells that are still attached in the wells. Cells can be gently triturated in the conical tubes by pipetting to obtain a single cell suspension.

The conical tubes can be centrifuged at 200 g for 5 min and the supernatant aspirated/discarded. The cells can be re-suspend in LIF-4i medium in a biosafety cabinet for cell counting using a hemocytometer or an automatic cell counter.

˜2×10⁵ cells can be plated into 2 mL of LIF-4i medium supplemented with 5 μM Y-27632 per well onto Vitronectin-XF-coated 6-well plates in a biosafety cabinet. The plate can be placed in a CO₂ incubator (5% O₂, 5% CO₂, humid atmosphere).

The next day, the plate can be gently swirled to lift all non-attached cells, the medium aspirated and replaced with 2 mL of LIF-4i medium in a biosafety cabinet daily for 2-3 days. The plate can be placed in a CO₂ incubator (5% O₂, 5% CO₂, humid atmosphere).

Note: After the 2nd and 3rd passages in LIF-4i using manual picking or the protocol steps detailed above, successful reversion will result into homogenous TIRN-hPSC LIF-4i cultures with only minimal residual differentiation carry-over. Stable LIF-4i cultures will tolerate bulk passaging thereafter.

Once homogeneous dome-shape colony cultures have been established (i.e., after the third passage in LIF-4i), cells bulk can be passaged every 2-3 days (FIG. 7A, 7B).

In a biosafety cabinet, culture medium can be discarded and each well of LIF-4i cultures washed by gently adding 2 mL of PBS. PBS can be discard and 1 mL of non-enzymatic EDTA-based dissociation buffer added. The cells can be incubated for 5 min at 37° C. in a CO₂ incubator (5% CO₂, humid atmosphere).

LIF-4i FF medium can be added (TABLE 1, at least 2-fold dilution) and the cells gently triturated by pipetting to recover all UTIRN-hiPSC in a single cell suspension. The cells can then be transferred into sterile 15 ml conical tubes. 1 mL of LIF-4i medium can be added to recover remaining cells in the well and combined UTIRN-hiPSC.

The conical tubes can be centrifuged at 200 g for 5 min and the supernatant aspirated/discarded. The cells can be re-suspend in LIF-4i medium in a biosafety cabinet for cell counting using a hemocytometer or an automatic cell counter.

˜2×10⁵ cells can be plated into 2 mL of LIF-4i medium supplemented with 5 μM Y-27632 per well onto Vitronectin-XF-coated 6-well plates in a biosafety cabinet. The plate can be placed in a CO₂ incubator (5% O₂, 5% CO₂, humid atmosphere).

The next day, the plate can be gently swirled to lift all non-attached cells, the medium aspirated and replaced with 2 mL of LIF-4i medium in a biosafety cabinet daily for 2-3 days. The plate can then be placed in a CO₂ incubator (5% O₂, 5% CO₂, humid atmosphere).

### 1.4 Cryopreservation, Thawing, and Use of LIF-4i XF/FF UTIRN-hiPSC

Typically, reverted conventional, primed hiPSC are expanded for at least 5-10 passages in LIF-4i FF, as indicated above, prior to use in functional studies or long-term cryopreservation. In general, the number of passages in conventional conditions and in LIF-4i FF conditions are recorded on each cryopreserved vial.

Excess LIF-4i FF UTIRN-hPSC not used in functional assays can be cryopreserved at each passage (TABLE 2), but freezing of lower post-reversion passages (e.g., <p5) will be hindered by low cell number and contamination from differentiated cells that may result in poor, or highly variable post-thaw recovery efficiencies.

In a biosafety cabinet, culture medium can be aspirated, cells washed in PBS (2 mL per well), PBS aspirated, and hPSC colonies dissociated into single cells using enzyme-free EDTA-based cell detachment solution (1 mL per well). The plate can then be placed for 5 min at 37° C. in a CO₂ incubator (5% CO₂, humid atmosphere).

The cell detachment solution can be diluted with LIF-4i medium (2-fold) in a biosafety cabinet and the TIRN-hPSCs collected in a sterile 15 mL conical tube.

The cells can be centrifuged at 200 g for 5 min and the cell pellet resuspended in LIF-4i medium (1-2 mL per well-equivalent) in a biosafety cabinet. The number of cells can be counted using a hemocytometer or an automatic cell counter.

UTIRN-hPSCs can be centrifuged in LIF-4i medium (200 g for 5 min) and the cells can be resuspend in a biosafety cabinet in freezing solution (TABLE 2), at a density of at least 1×10⁶ cells/mL.

The cells can be transferred into long-term storage cryogenic tubes and placed into a slow-freezing container. The samples can be allowed to freeze overnight in a −80° C. freezer.

The next day, the cryovials can be transferred into a liquid nitrogen freezer for long term storage.

For thawing, the frozen vial can be placed into a 37° C. water bath for ˜2 min. The vial can be sterilized (i.e., ethanol spray), hPSCs transferred in a sterile 15 ml conical and the cells slowly diluted 10-fold in LIF-4i medium (TABLE 1) supplemented with 5 μM of Rho-associated protein kinase (ROCK) inhibitor Y-27632 within a sterile biological safety hood cabinet.

The cells can be centrifuged at 200 g for 5 min. In a biosafety cabinet, cell-free supernatant can be discarded and the cell pellet resuspended in LIF-4i medium (1-2 mL) supplemented with 5 μM ROCK inhibitor Y-27632.

Exclusion of Y-27632 will generally result in poor post-thawing recovery efficiencies.

The thawed cells resuspended in LIF-4i/ROCK Inhibitor can be transferred onto Vitronectin-XF-coated wells. LIF-4i cultures are routinely cryopreserved at a density of 1×10⁶ cells per vial. Each of these vials is thawed in one well of a Vitronectin-XF-coated 6-well plate.

The next day, regular LIF-4i FF UTIRN-hiPSC expansion can be started.

### 1.5 Validation of Genomic Integrity and Retention of Parental Imprints or LIF-4i FF-Reverted TIRN-hPSC Prior to Use in Functional Assays

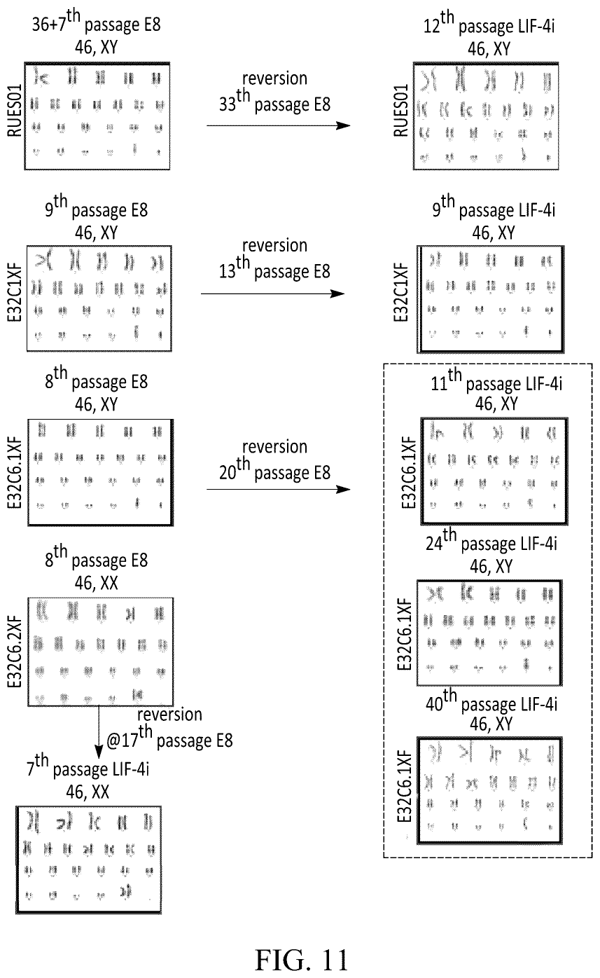

The starting primed, conventional hPSC cultures can be screened for possession of a normal karyotype (e.g., with Giemsa-band staining analysis using methods known in the art) before initiating LIF-4i reversion, to eliminate conventional hPSC populations that may harbor abnormal genomic alterations that may drive artefactual selective survival advantage in clonal LIF-4i FF conditions (FIG. 11).

Conventional hPSC cultures can be freshly reverted to a naïve-like state with LIF-4i several weeks prior to their use in functional studies or directed differentiations.

Routine prolonged ‘maintenance’ culture in LIF-4i FF conditions for more than 10 passages following naïve reversion is not recommended. Routine expansion and maintenance of hESC and hiPSC lines should be performed using conventional culture systems (e.g., in E8, or MEF/hESC medium with bFGF).

Post-reverted TIRN-hPSC lines can be assessed for retention of normal karyotypes 5-7 passages after LIF-4i FF reversion (e.g., with Giemsa-band staining analysis, or other method of choice, FIG. 11).

All reverted TIRN-hPSC lines can be assessed for retention of normal parental genomic imprints by a DNA methylation analysis of choice (e.g., protocols for CpG DNA microarray analysis of parental imprints in LIF-3i-reverted TIRN-hPSC are known in the art) after 5-10 passages of LIF-4i FF TIRN reversion.

### 1.6 General Experimental Design Guidelines for Differentiation of UTIRN-hiPSC

LIF-4i FF UTIRN-hiPSCs can be directly utilized in established directed differentiation protocols without any extended cell culture manipulations. For example, but not by way of limitation, “re-priming” (i.e., converting TIRN-hPSC back to conventional primed conditions prior to their use in directed differentiation assays) is not necessary with the LIF-4i FF methods. However, the PKC inhibitor Go6983 requires washing out prior to starting differentiation protocols (successful differentiation has also resulted with additional removal of the MEK inhibitor PD0325901). LIF-4i FF TIRN-hPSC are still capable to differentiate directly without washing Go6983, but the presence of the PKC inhibitor and its intracellular retention can interfere with directed differentiation protocols. The medium can be switched to LIF-4i without PKC inhibitor for at least one hour (intracellular retention of the small molecule Go6983 is easily detectable in a red channel by flow cytometry without any additional staining).

To control for the impacts of assay and interline variability in the functional testing of individual hPSC lines, in certain embodiments, cross-validation of lineage-specific differentiation potencies can be ensured by employing independent differentiation protocols with at least three hPSC lines derived from independent genetic backgrounds (i.e., multiple donor-derived hiPSC and hESC).

For functional comparisons of conventional vs UTIRN-hiPSC, parallel sibling cultures can, in certain embodiments, be employed, at equivalent passage number, and from the same (isogenic) hPSC line in parallel between conventional lineage-primed and LIF-3i-reverted hPSC cultures. In such embodiments, it can be useful to maintain primed/naïve sibling isogenic hPSC cultures in parallel in their respective media (e.g., E8vs. LIF-4i FF), and simultaneously differentiate in parallel using identical differentiation protocols and materials, to eliminate experimental bias.

For isogenic primed vs. TIRN hPSC comparisons, it can, in certain embodiments, be useful to adjust initial plating densities for each individual differentiation assay. Suitable detailed protocols for neural progenitor, definitive endoderm and hemato-endothelial directed differentiations of LIF-4i FF-reverted TIRN-hPSC are known in the art.

LIF-4i-reverted UTIRN-hiPSC can exhibit a more robust proliferative and differentiation capacity in directed differentiation assays than conventional hPSC. UTIRN-hiPSC typically require a lower initial plating concentration than conventional hPSC, and unlike their conventional primed hPSC counterparts, typically do not require the use of anti-apoptotic reagents to enhance their clonal survival following enzymatic digestion in differentiation assays.

### Example 2: Characterization and Validation of the Phenotypic, Molecular, and Functional Pluripotencies of LIF-4i FF-Reverted hPSC

### 2.1 Colony Morphologies

The transition between primed, conventional, and LIF-4i FF TIRN culture systems is accompanied by distinct physical changes in hPSC colony morphology. Conventional hPSC cells proliferate as flat, wide monolayer colonies that expand rapidly from small cell clumps (on MEF or feeder-free conditions), but poorly as single cells. Exposure of conventional hPSC lines to LIF-4i promotes the growth and expansion and of smaller, tightly-packed, dome-shaped colonies that arise clonally from single cells. These morphological changes are completely reversible, and LIF-4i-reverted dome-shaped colonies can spontaneously transition back to a conventional monolayer morphology if LIF-4i FF is withdrawn and cells are re-cultured in standard conventional hESC medium supplemented with bFGF. Additionally, expansion of LIF-4i FF-reverted cells at high confluent densities (or prolonged culture without frequent passaging) results in spontaneous reacquisition of the flat, conventional morphology with reduced clonal efficiency; emphasizing the need for diligent maintenance and care of LIF-4i FF-reverted hPSC (e.g., <40-60% confluence).

FIG. 7A outlines the transition between primed (i.e. E8) and the LIF-4i FF-TIRN-reverted system. Upon passaging into LIF-4i, hPSC immediately adopt a smaller dome-shaped morphology that expands from single cells. Photomicrographs document the morphology of the hiPSC line 6.2 during the first passages into LIF-4i (bulk passaging without colony picking) and after stable expansion for 10 consecutive passages (FIG. 7B). To promote bulk passaging, rigorous attention must be observed to maintain conventional, primed cultures at reduced density (FIG. 9A, 9B) and with minimal carry-over of spontaneous differentiation, if any. Suboptimal conditions or distinct hPSC lines will reduce initial clonal efficiencies or will promote emergence of differentiation in parallel to naïve colonies (FIG. 10). In this case, differentiated cells may only be evident during the 2^{nd} passage and efforts should be made to minimize any carry over. The LIF-4i FF TIRN culture system requires overnight supplementation with the ROCK inhibitor Y27632 after passaging. Non-inclusion of ROCK inhibitor will result into suboptimal cell survival and attachment (FIG. 9C).

### 2.2 Pluripotency Marker Expression by qRT-PCR, Immunofluorescence Staining, and Flow Cytometric Analysis

Evaluation of pluripotency markers during the transition from conventional to a naïve-like UTIRN-hiPSC state following continuous LIF-4i FF culture can be monitored by live antibody staining without detecting negative effects on UTIRN-hiPSC expansion (e.g., live-staining fluorochrome-conjugated antibodies against TRA-1-81, TRA-1-60, and SSEA4).

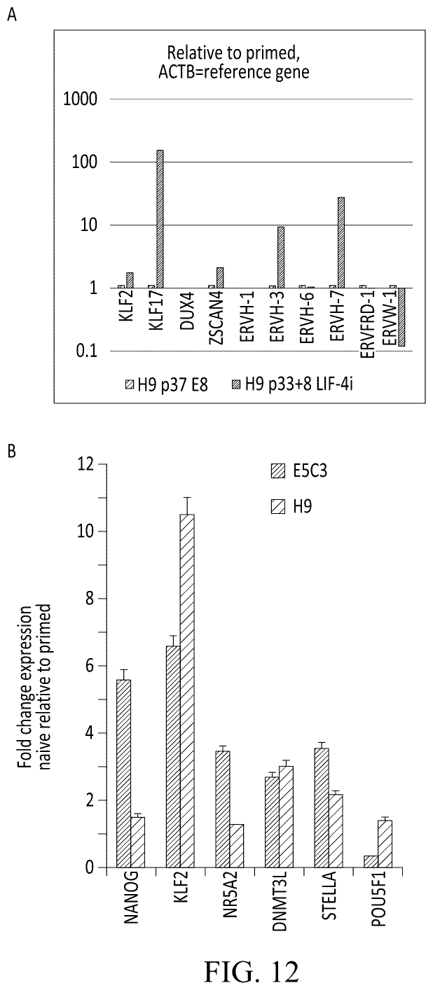

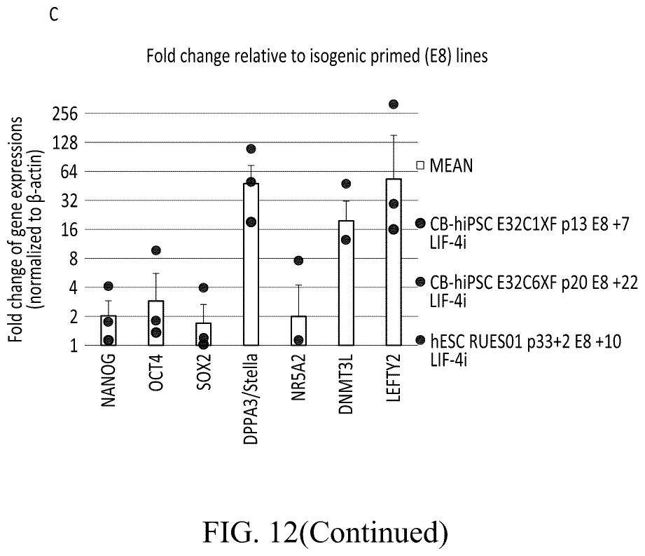

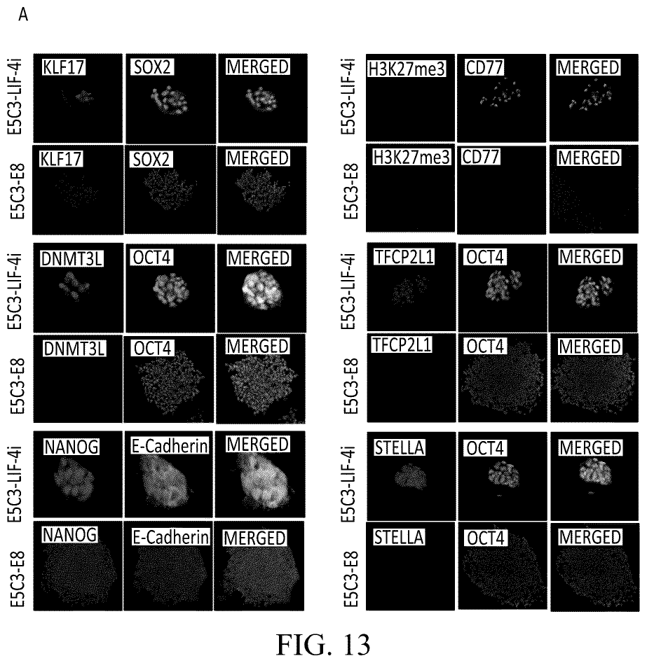

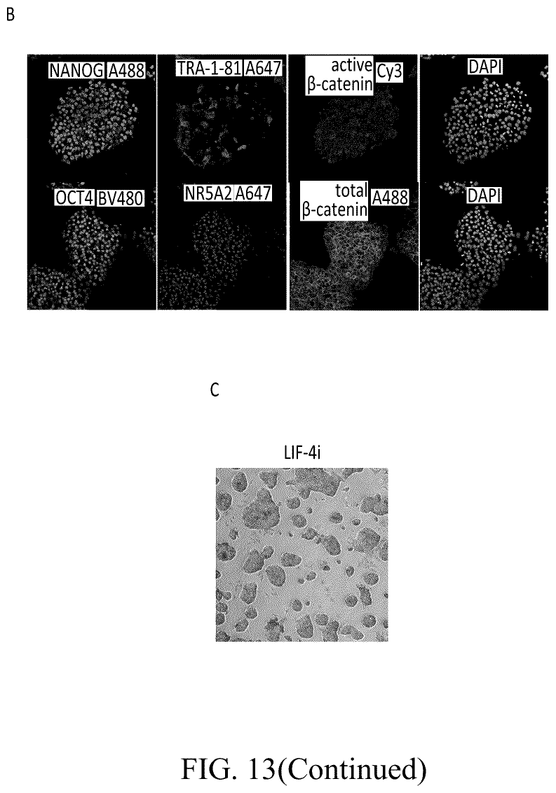

Retention of naïve pluripotency during LIF-4i FF TIRN reversion can also be routinely monitored by using qRT-PCR of naïve-specific transcripts (FIG. 12) or flow cytometric analysis of pluripotency-associated surface markers (FIG. 7, FIG. 8). Expression of TRA-1 and SSEA antigens and other proteins was confirmed by immunofluorescence of intact, fixed colonies in situ (FIG. 13). The levels of these markers inversely correlate with the frequency of spontaneous differentiation that may occur when transitioning from conventional hPSC to TIRN-hPSC conditions. Additional surface antigens that may more specifically mark human naïve-like states in vitro can also be employed to detect effective TIRN-hPSC reversion (FIG. 13).

LIF-4i FF cultures maintain similar expressions of TRA-1-81, TRA-1-60, and SSEA4 to those observed in conventional and LIF-3i/MEF conditions. The retention of these markers can be routinely monitored by flow cytometric analysis. Results are presented for the hiPSC line 6.2 (FIG. 7C). However, due to intrinsic red fluorescence of the small molecule Go6983, specific efforts should be made to either avoid red fluorescence channels or to thoroughly wash the cells before acquisition in LIF-4i FF conditions. Cells that have been expanded in LIF-4i can be incubated in LIF-4i TIRN medium that does not include Go6983 for short periods of time to promote exclusion of the small molecule from the cells and reduce auto-fluorescence for flow cytometry analysis.

### 2.3 Validation of Molecular Naïve Pluripotency of UTIRN-hiPSC

Because the genetic background of hPSC lines has been characterized as a strong contributor to interline variability, it is important to rigorously assess isogenic cultures at matching culture time points when comparing conventional hiPSC to TIRN-hiPSC culture systems (FIG. 6). Since some of these systems have already been shown to generate hPSC populations with aberrant genomic and epigenetic configurations, any naïve reversion method should be assayed with a number of TIRN-hiPSC of independent genetic backgrounds, in a manner that is sufficient to validate biological reproducibility and exclude non-developmentally relevant “pseudo-pluripotent” states (i.e., with apparent hallmarks of molecular pluripotency but lacking functional differentiation abilities). Zimmerlin et al., Development 2016; 143(22): 4368-4380 further extended the validation of the LIF-3i culture system to include assaying the molecular and functional pluripotencies of reverted TIRN-hiPSC derived from various reprogramming methods, which is another known putative contributor of functional variability between pluripotent states. Similar to the LIF-3i/MEF naïve method, the LIF-4i TIRN culture method was successfully applied to four hESC lines (RUES01, male, RUES02, female RUES03, male, H9, female), one 7-factor episomal stroma-primed non-XF CB-hiPSC (6.2, female), one 4-factor episomal stroma-primed non-XF CB-hiPSC (E5C3, female), four 4-factor episomal stroma-primed XF CB-hiPSC (E32C1XF, male, E32C4XF, male, E32C6.1XF, male, E32C6.2XF, female), one 7-factor episomal adult fibroblast-hiPSC (C2, female).

Accordingly, most studies of human naïve culture systems have focused on assaying molecular pluripotency of TIRN-hPSC at 1) the epigenetic level (e.g., histone marks by ChIP sequencing or ChIP-PCR, global DNA methylation by immunoblots or whole genomic bisulfite sequencing, allele-specific CpG methylation microarrays, OCT4enhancer predominant usage by reporter systems, global activity at regulatory elements by DNAse I hypersensitivity, and repeat clement profiling by RNA-sequencing), 2) transcriptomic level (RNA-sequencing, expression microarrays, and quantitative RT-PCR), protein expression analysis (e.g., FACS, immunofluorescent microscopy, and Western blotting) and 3) via metabolic studies (e.g., glycolysis, oxidative phosphorylation and nicotinamide metabolism).

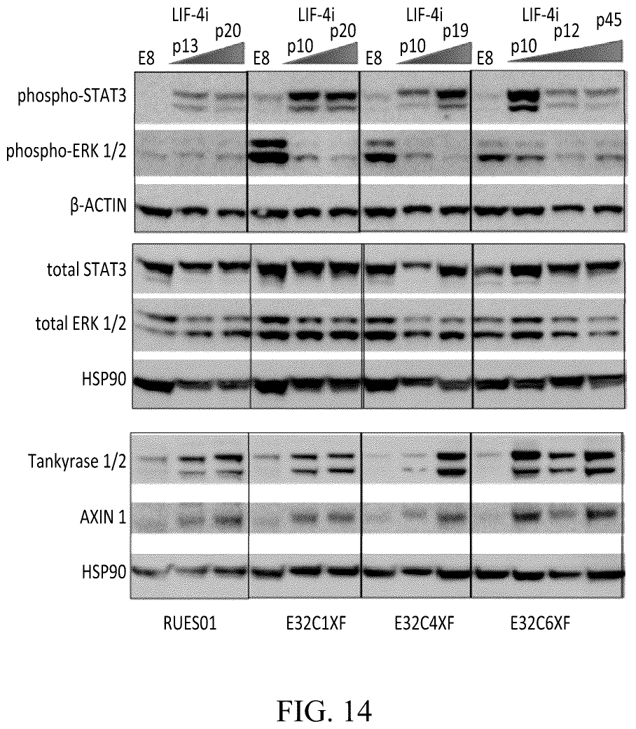

Representative examples of immunofluorescence stains and Western blot detection of expression for key markers of molecular pluripotency are shown for non-XF and XF hPSC lines (FIG. 13 and FIG. 14 for LIF-4i FF TIRN systems). For example, FIG. 14 shows levels of expression for the activated phosphorylated (phospho) and total isoforms of STAT3 and ERK1/2, which are key molecular hallmarks of mouse ESC-like naïve pluripotency for three XF-derived FF hiPSC and one control hESC lines. These were detected using anti-STAT3 and anti-ERK1/2 primary antibodies.

LIF-4i FF colonies maintained uniform expression of the pluripotency markers NANOG, OCT4 and TRA-1-81, acquire expression of naïve markers (i.e., NR5A2) and display elevated levels of the activated isoform of beta-catenin (FIG. 13), similar to the LIF-3i/MEF culture system. We present Western blot data (FIG. 14) for 5representative hPSC cells (two hESC lines, H9 and RUES01) and three CB-derived XF/FF UTIRN-hiPSC lines that were derived in the Zambidis lab E32C1XF, E32C4XF and E32C6XF) as described above (FIG. 5). Results are shown for multiple passages and show stable expression of the phosphorylated activated isoform of STAT3, with concomitant reduction of phosphorylated active ERK1/2.

The data also further illustrate that the LIF-4i FF system reproduces mechanistic regulation of active beta-catenin via tankyrase inhibition, as previously described for the LIF-3i/MEF culture system (FIG. 13B). The tankyrase/PARP inhibitor XAV939 interrupts tankyrase-mediated PARylation and stabilizes multiple tankyrase targets (i.e., tankyrase, Axin1) that would be normally subject for proteolytic degradation upon PARP activity. We previously reported the importance for Axin stabilization to further reinforce active beta-catenin regulation in presence of the GSK3β inhibitor CHIR99021.

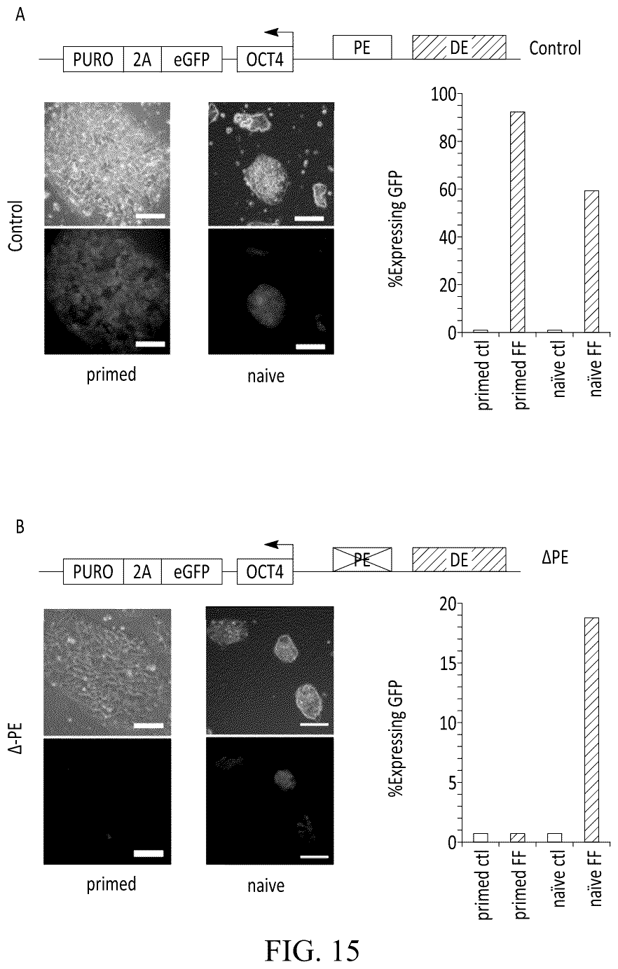

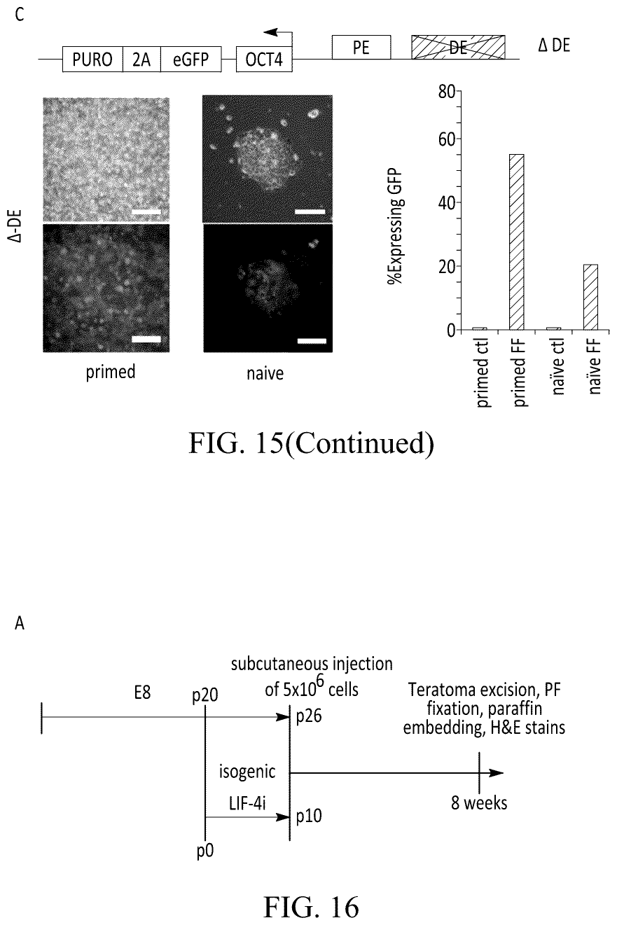

Additional evidence of molecular naïve pluripotency of UTIRN-hiPSC was provided by evaluating the epigenetic functionality of the OCT4 promoter in conventional, primed vs UTIRN-hiPSC (FIG. 15). We assayed for the activation of the proximal (PE) and distal (DE) enhancers of the OCT4 promoter in LIF-3i-reverted vs. primed hPSC. Using luciferase reporter assays and stable transgenic genomic DE/PE sequence mutant reporter hPSC lines, we demonstrated that LIF-4i reversion potentiated naïve ESC-like activation of the DE of the OCT4 promoter (FIG. 15B), while primed hPSCs displayed exclusive mEpiSC-like PE OCT4 activity (FIG. 15C).

### 2.4 Evaluation of Functional Pluripotency of UTIRN-hiPSC

The most rigorous assay of functional pluripotency of PSC is the blastocyst injection chimera assay, which is limited in the testing of UTIRN-hPSC lines for ethical reasons. Alternatively, several groups that reported the generation of TIRN hPSC derived with various other methods have also attempted to generate interspecies chimeras. However, these attempts have yielded extremely low or unsuccessful contribution of differentiated TIRN-hPSC lineages to murine or porcine embryos, in comparison to the chimera-generating capacity of standard mouse ESC. Additional functional studies have investigated directed in vitro differentiation of putative TIRN-hPSC derived via various methods, but have revealed biased, defective, or diminished multi-lineage differentiation capacity, with concomitant harboring of epigenetic abnormalities. Similar epigenomic aberrations, especially at imprinted loci, have been detected in mouse ESC following prolonged exposure to the LIF-2i cocktail. Interestingly, some culture systems that included the non-specific tankyrase/PARP inhibitor XAV939 have reported global improvements in specific attributes of functional pluripotency of PSC, such as improved chimera contribution and an enhanced capacity for in vivo trophectoderm contribution.

An isogenic approach can be employed to compare directed differentiation to ectodermal, mesodermal, and endodermal lineages hPSC in primed (i.e., E8) vs. feeder-free naïve LIF-4i FF TIRN conditions. However, initial plating density and conditions should be adjusted for each individual assay. Additionally, certain LIF-4i FF protocols are compatible with rapid in-bulk reversion of multiple primed hPSC lines.

Using a broad collection of independently-derived UTIRN-hPSC, multilineage differentiation assays have been employed to show that the LIF-4i FF system dramatically improved the functional pluripotency of conventional, primed hPSC lines. In addition, a systematic analysis of conventional hiPSC vs UTIRN-hiPSC lines in isogenic pairs has been performed to eliminate interline-dependent variations. UTIRN-hiPSC lines do not require a re-priming step prior to EB differentiation. However, UTIRN-hiPSC proliferate at significantly higher clonal rates than isogenic cells expanded in E8, and thus, initial lower plating densities require adjustment to allow each culture to reach confluence at a similar time point.

Investigators can utilize multiple assays to demonstrate improved functionality of LIF-4i FF-reverted UTIRN-hiPSC, including not only in vivo teratoma assays, but also in vitro directed differentiation assays to neural (ectodermal), definitive endoderm and hematovascular (mesodermal) lineages using multiple assays (e.g., 2D APEL 2 and 3D embryoid body systems). To control for assay-dependent reproducibility, at least two different differentiation methods should typically be performed in replicate for each isogenic pair of primed LIF-4i FF hPSC cultures. The experimental design should include a robust number (e.g., >3-5) primed LIF-4i FF isogenic pairs of hPSC lines from multiple, independent donor genetic backgrounds.

LIF-4i FF cultures do not promote spontaneously-arising chromosomal defects as was reported in other human naïve reversion systems. Nevertheless, the use of low passage LIF-4i FF hPSC for functional assays (i.e., directed differentiations) for prevention of genomic aberrations that may arise from prolonged clonal culture is recommended. During the performance of functional assays, primed cultures should typically be maintained in parallel conditions and differentiated using the same materials and methods as isogenic TIRN-hPSC, to eliminate potential experimental bias. Excluding the protein kinase C inhibitor from LIF-4i FF TIRN cultures for at least one hour at the initiation of directed differentiation protocols is also recommended. Initial plating densities should also be adjusted to allow each culture to reach confluence at a similar time point.

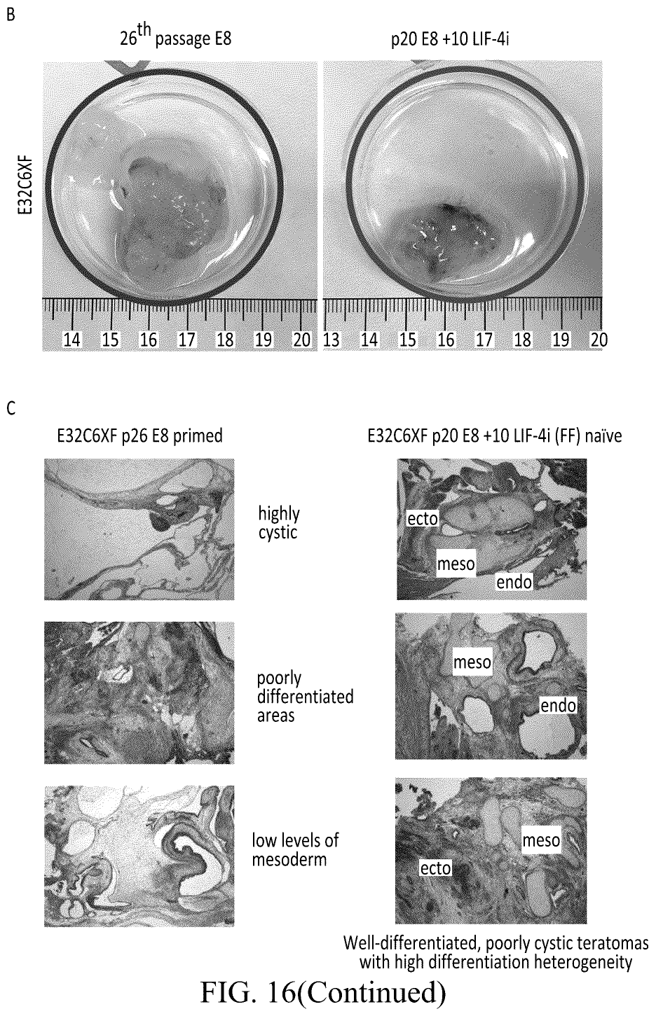

Teratoma Assays. Functional pluripotency of UTIRN-hiPSC was assayed using teratoma differentiation protocols, without any requirement for re-priming LIF-4i FF hPSC before injection into animals (FIG. 16A). Injection of identical numbers of cells from isogenic feeder-free primed E8 and naïve LIF-4i FF cell lines (representative results are shown for the XF/FF CB-hiPSC line E32C6XF) revealed gross disparities between primed, conventional and UTIRN-hiPSC eight weeks post-injection (FIG. 16B). While primed E8 E32C6XF cells displayed strong histological bias to definitive endoderm with minimal mesoderm and non-detectable well-differentiated neuro-ectoderm tissues, reverted UTIRN-hiPSC (after 10 passages in LIF-4i) exhibited dramatically more robust areas of differentiated cells for all three germ layers (FIG. 16C).

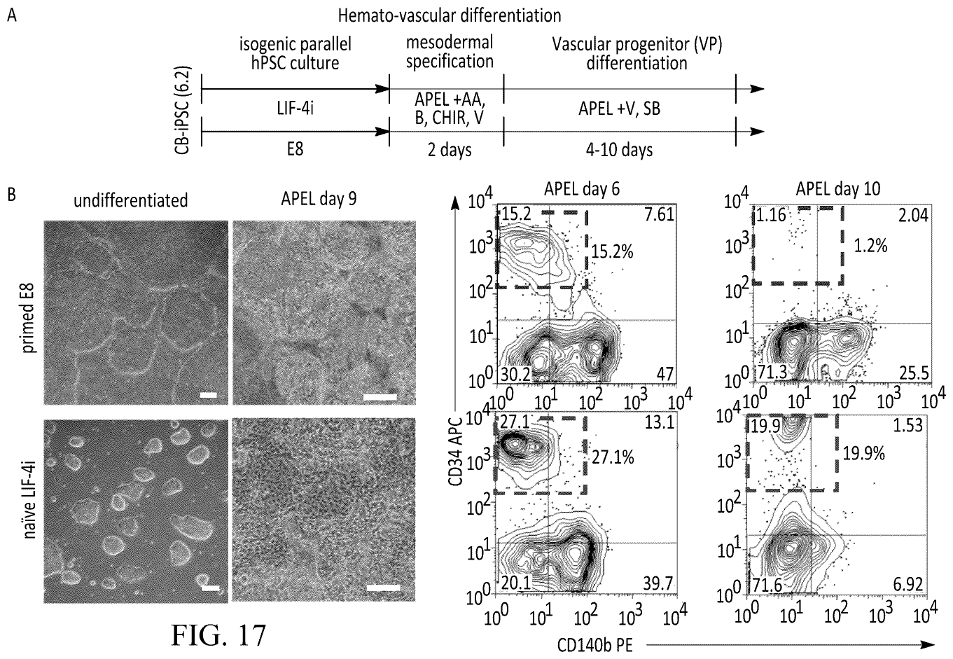

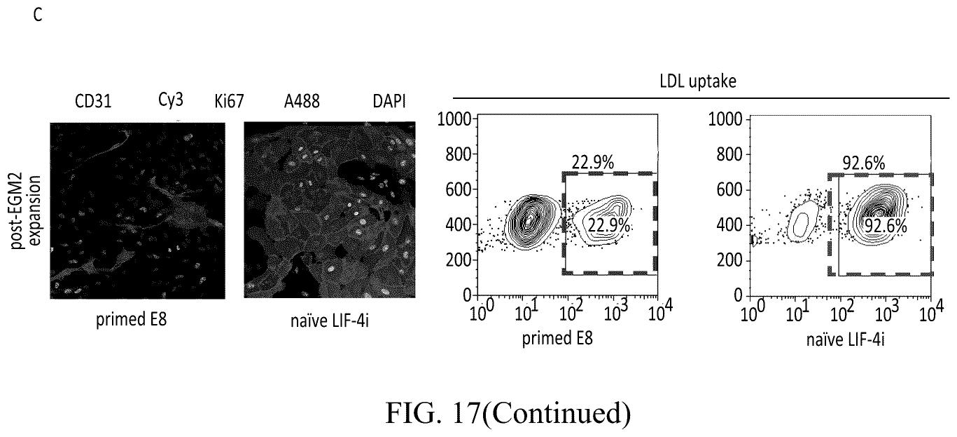

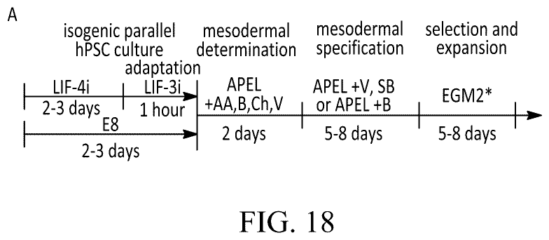

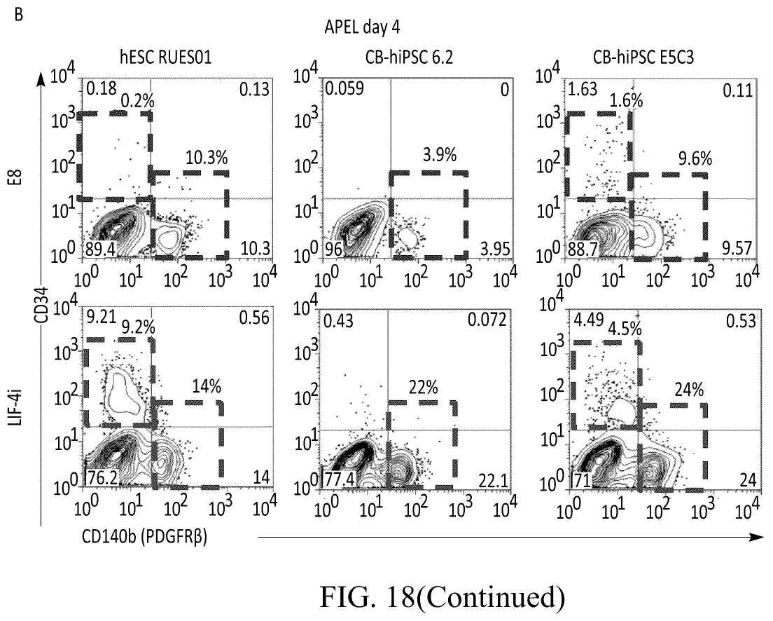

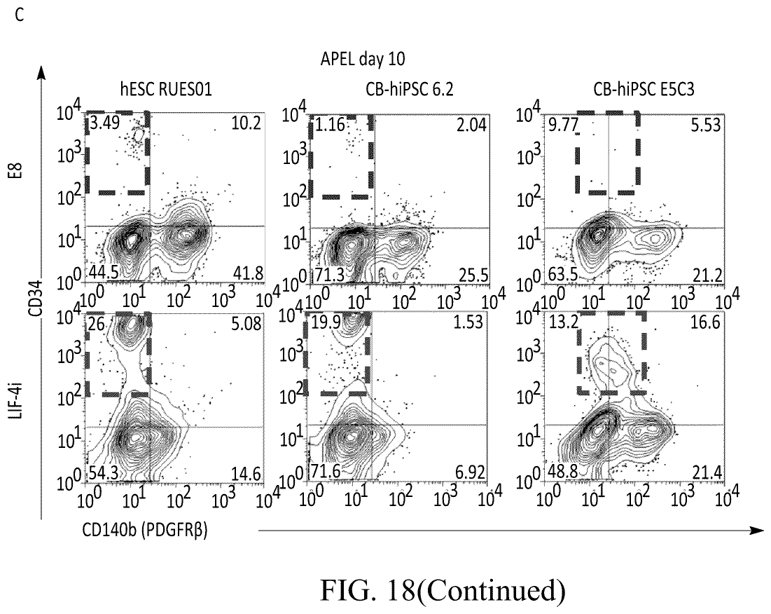

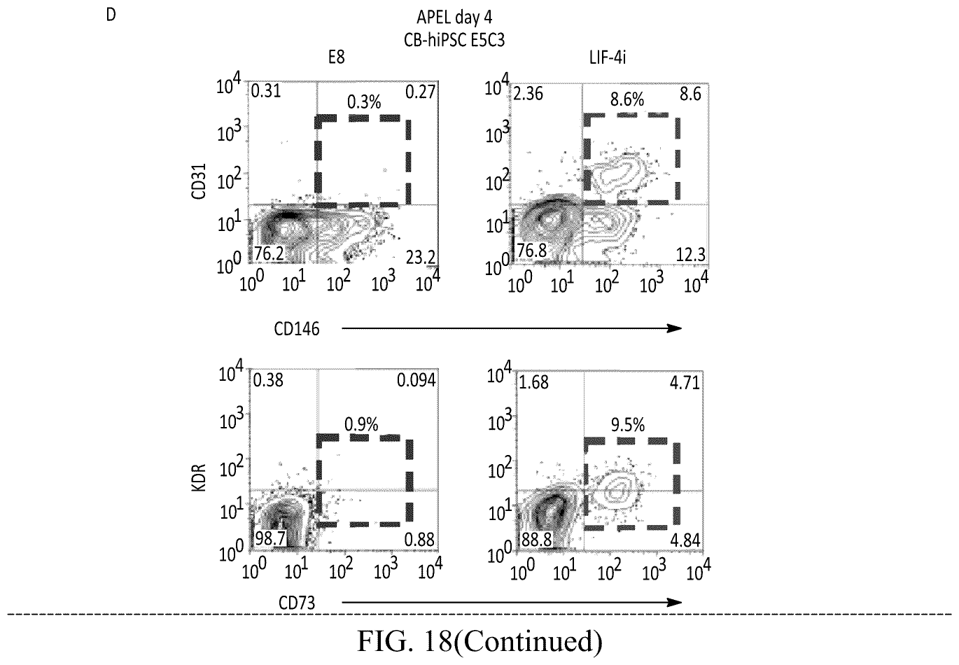

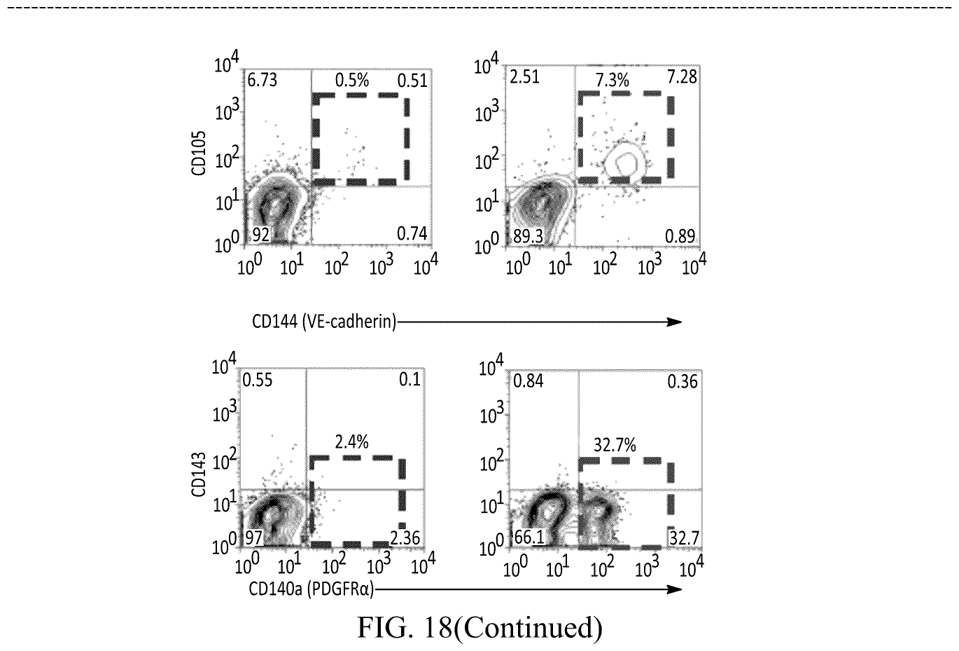

In Vitro Directed Vascular Differentiation. LIF-4i FF UTIRN-hPSC cultures routinely demonstrated more robust differentiation capacities than their isogenic primed hPSC counterparts in not only teratoma assays, but also in XF directed differentiation assays (APEL system). For example, directed UTIRN-hiPSC differentiation from LIF-4i in XF vascular lineage differentiation conditions resulted in more rapid kinetics of pericytic vascular progenitor (VP; CD31+CD146+) and endothelial progenitors (CD34+CD140b+), and other mesodermal vascular populations (KDR+, CD144+) than from their isogenic conventional hPSC counterparts (FIG. 17, FIG. 18). These UTIRN-hiPSC cultures generated overall higher frequencies of cobblestone endothelial cell monolayers that were enriched in CD31+VP possessing higher proliferative rates (e.g., Ki-67+) and LDL-binding endothelial functionality (e.g., Ac-Dil-LDL, UEA1+, FIG. 17C).

### 2.5 Genomic Integrity Studies: Assessment of DNA Double-Strand Repair Mechanisms in UTIRN-hiPSC

Preserving genomic stability is critical during early embryonic development. Non-homologous end joining (NHEJ) and homology-directed repair (HDR) are the main mechanisms that eukaryotic cells use for double-strand DNA break (DSB) repair and preservation of genomic integrity. Pre-implantation embryos and mouse embryonic stem cells (mESC) favor the more accurate HDR rather than the fast, but error-prone NHEJ. As a result, mESC exhibit poor efficiency for rejoining radiation-induced DNA double-strand breaks in comparison to human primed ESC that similarly to mouse post-implantation embryos predominantly utilize NHEJ for DSB repair. Various strategies have been recently developed to overcome limited precise editing in CRISPR-Cas9-mediated genome editing, including altering HDR or NHEJ machinery. The predominant DSB repair strategy in naïve hPSC remains unclear, but superior HDR efficiencies would be expected if naïve hPSC adopt developmental expressions that are closer to preimplantation embryos. However, HDR efficiencies were actually impaired using the human naïve method 4iLA, which also exhibits elevated aneuploidy frequency and erased parental imprints.

LIF-4i stably reverts conventional, primed hPSC to a functional human naïve epiblast-like state that recapitulates molecular and epigenetic signatures of the human pre-implantation epiblast. More importantly, UTIRN-hPSC maintained normal karyotypes and epigenomic imprints. If UTIRN-hPSC possess improved HDR, this would greatly impact developmental biology and regenerative medicine. While mESC may harbor deficiencies for NHEJ and radiation-induced DSB repair, retention of NHEJ in UTIRN-hPSC would also be important to support efficient and rapid repair in DSB-inducing environment. The study of HDR/NHEJ mechanisms in UTIRN-hiPSC may functionally validate the DSB repair strategies utilized by UTIRN-hPSC. Thus, the HDR machinery of LIF-3i-reverted TIRN-hPSC vs. their isogenic conventional, primed hPSC counterparts was investigated.

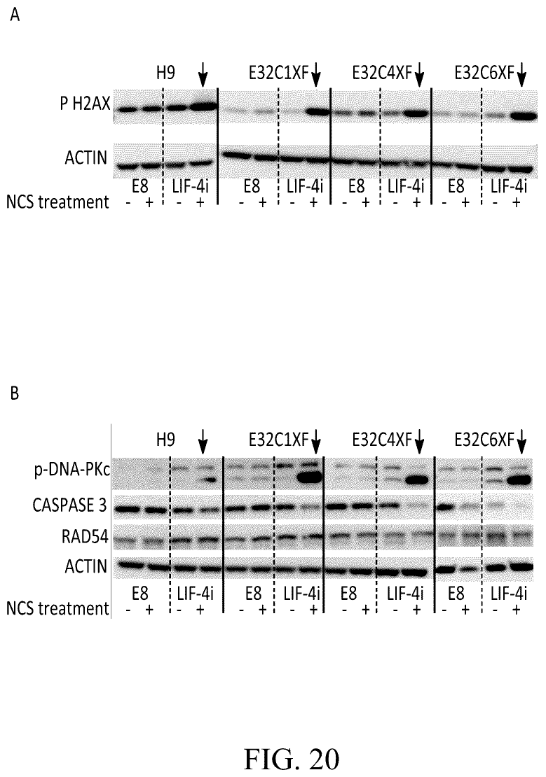

Methods. Conventional (primed) XF CB-hiPSC lines and control H9 hESC were grown in E8 medium. Isogenic TIRN-hPSC were cultured in parallel using LIF-4i. For Western blotting, whole cell lysates were prepared in RIPA buffer, 1.5 mM EDTA with protease inhibitor. Protein lysates were quantified using the BCA assay. Equal amounts of total protein lysate (e.g., 20 μg) were loaded into precast polyacrylamide gradient gels, size-separated using the Mini-PROTEAN electrophoresis system and blotted onto PDVF membranes for chemiluminescent analysis. Primed hPSC and isogenic TIRN-hPSC were assayed for endogenous DNA damage during routine culture (FIG. 20) or upon DSB induction using the radiomimetic agent neocarzinostatin (NCS). In these experiments, hPSC were treated for 4 hours with NCS before preparing lysates.

Results. To explore the effects of LIF-4i on DSB repair, the levels of major components of NHEJ and HDR were evaluated, particularly the catalytic subunit of DNA-PK and BRCA1. which are two critical components of the NHEJ and HDR machineries respectively, and that are both regulated by both PARP1 and tankyrase PARylating and non-PARylating activities. The tankyrase inhibitor XAV939 has been shown to not only downregulate tankyrase PARylating activity, but also PARP1-mediated PARylation in 293T cells. Inhibition of PARP1-mediated PARylation has been shown to not only promote DNAPK auto-phosphorylation and activation, but also controls and activates BRCA1-mediated HDR. Tankyrase knock-down or XAV939 inhibition increases DNAPKc activity. Alternatively, tankyrase controls the activity of BRCA1 complexes and HDR by interacting with the DNA damage sensor MDC1. MDC1 is a DNA checkpoint protein that is essential to spread the DNA response machinery and that is cleaved by caspase3 during apoptosis. This tankyrase activity on MDC1 is independent of tankyrase-mediated PARylation and is not diminished in the presence of the tankyrase/PARP inhibitor XAV939 in the 293T cell line.

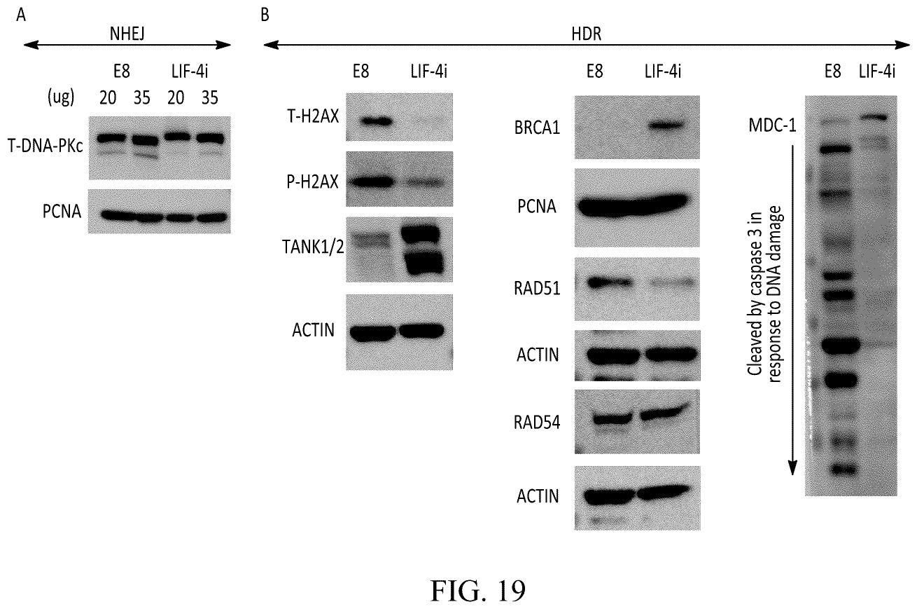

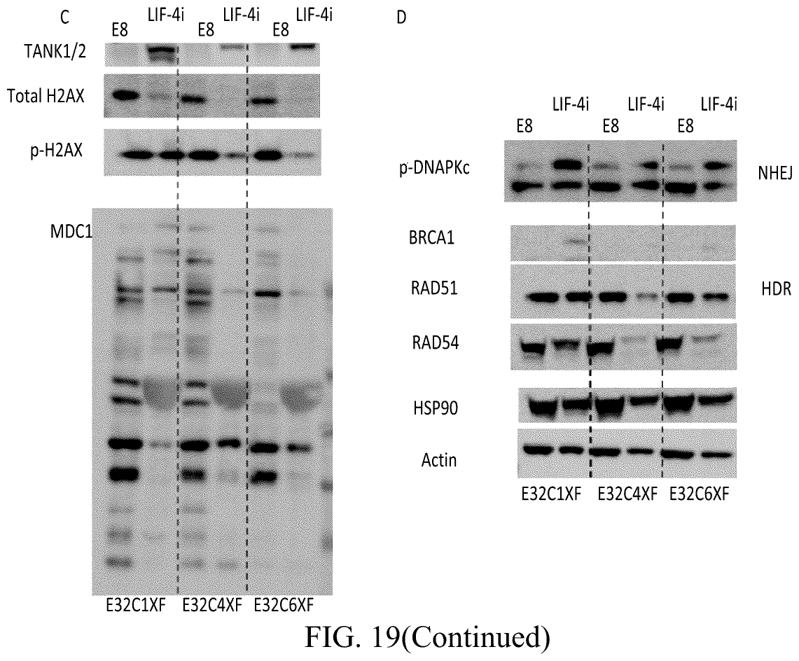

Unlike mESC, UTIRN-PSC that were grown in LIF-4i continued to express high levels of DNA-PKc (FIG. 19A), which would be required to ensure efficient NHEJ-based DSB repair, especially under stressful culture conditions. In accordance with these results, LIF-4i TIRN-hPSC displayed low endogenous levels of H2AX and phosphorylated H2AX (FIG. 19B), in contrast with isogenic parallel E8 cultures, that appeared to accumulate higher levels of DSB. In agreement with previous observations, tankyrase auto-PARylation and proteolysis is inhibited by XAV939 and tankyrase proteins accumulate in LIF-4i (FIG. 19C). These high levels of tankyrase protein correlated with higher stability of full length MDC1 and detectable levels of BRCA1 FIG. 19C, 19D). These results support the hypothesis that LIF-4i TIRN culture promotes competence for HDR, even though they also maintain NHEJ machinery and efficiently preserve genome integrity with low levels of DSB (which was revealed by reduced amounts of phospho-H2AX). These effects were reproducibly detectable in three XF/FF UTIRN-hiPSC.

Previous studies suggested that mESC were deficient in repairing radiation-induced DSB. Consequently, whether reduced H2AX levels in LIF-4i UTIRN-hiPSC reflected a DSB repair deficiency rather than efficient repair in endogenous condition was investigated. Primed E8 and LIF-4i UTIRN-hiPSC were exposed to the radiomimetic neocarzinostatin to induce DSB (FIG. 20A-20B). All TIRN-hPSC responded dramatically to NCS with elevated levels of phospho-H2AX surpassing significantly the response of primed E8 hPSC (FIG. 20A). A strong increase of the levels of phosphorylated active DNA-PKc were detected, indicating that in response to NCS-induced DNA damage UTIRN-hiPSC rapidly activated the NHEJ response, resulting into lower levels of caspase 3, which further indicates superior resistance to DNA damage (FIG. 20B).

These studies provide evidence that UTIRN-PSC can efficiently employ HDR as a DSB repair strategy, yet remain competent to repair radiation-induced DSB. The inclusion of XAV939 in LIF-3i appeared to interrupt tankyrase auto-PARylation-mediated proteolysis. Elevated tankyrase protein in TIRN-hPSC correlated with stabilization of the non-cleaved isoform of its partner MDC1 and reinforcement of RAD54 and BRCA1.Accordingly the data described herein indicates that modifying the balance between NHEJ and HDR events by manipulating naïve vs. primed pluripotent states can enhance genome editing strategies (e.g., CRISPR-Cas9, plasmid-based HDR), and allow for more facile gene targeting of hPSC.

The contents of all figures and all references, patents and published patent applications and Accession numbers cited throughout this application are expressly incorporated herein by reference.

What is claimed is:

1. A method for generating a population of therapeutic progenitor cells under clinical grade, current good manufacturing practice (cGMP)-compliant conditions, comprising:
(a) contacting a population of human induced pluripotent stem cells (hiPSCs) with a tankyrase/PARP-inhibitor-containing composition comprising:
(i) leukemia inhibitory factor (LIF);
(ii) a Glycogen Synthase Kinase 3-β (GSK3B) signaling pathway inhibitor;
(iii) a mitogen-activated protein kinase (MEK) signaling pathway inhibitor;
(iv) a tankyrase/PARP inhibitor; and
(v) a protein kinase C (PKC) inhibitor;
(b) performing feeder-free (FF) and xeno-free (XF) culture of the hiPSCs to generate a population of these therapeutic progenitor cells under clinical grade, cGMP-compliant conditions.

2. The method of claim 1, wherein the tankyrase/PARP inhibitor is selected from: XAV939, IWR-1, G007-LK, JW55, AZ1366, JW 74, NVP-TNKS656 and combinations thereof.

3. The method of claim 1, wherein the GSK3β signaling pathway inhibitor is selected from: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR 99021), 5-Ethyl-7,8-dimethoxy-1H-pyrrolo[3,4-c]isoquinoline-1,3(2H)-dione (3F8), 1-(7-Methoxyquinolin-4-yl)-3-[6-(trifluoromethyl)pyridin-2-yl]urea (A 1070722), N6-[2-[[4-(2,4-Dichlorophenyl)-5-(1H-imidazol-1-yl)-2-pyrimidinyl]amino]ethyl]-3-nitro-2,6-pyridinediamine (CHIR 98014), lithium chloride (LiCl), 4-benzyl-2-methyl-1,2,4-thiadiazolidine-3,5-dione (TDZD-8), 5-iodo-indirubin-3′-monoxime (I3′M), N-(4-methoxybenzyl)-N′-(5-nitro-1,3-thiazol-2-yl)urea (AR-A014418) and combinations thereof.

4. The method of claim 1, wherein the MEK signaling pathway inhibitor is selected from: PD032590, CI-1040 (PD184352), cobimetinib (GDC-0973, XL518), Selumetinib (AZD6244), MEK162, AZD8330, TAK-733, GDC-0623, Refametinib (RDEA119; BAY 869766), Pimasertib (AS703026), RO4987655 (CH4987655), RO5126766, WX-554, HL-085 and combinations thereof.

5. The method of claim 1, wherein PKC inhibitor is Go6983.

6. The method of claim 1, wherein the hiPSCs are derived from primed hiPSCs.

7. The method of claim 6, wherein the priming of hiPSCs comprises contacting the hiPSCs with a serum replacer-containing media.

8. The method of claim 6, wherein priming the hiPSCs comprises contacting the hiPSCs with a ROCK inhibitor.

9. The method of claim 8, wherein the ROCK inhibitor is Y-27632.

10. The method of claim 1, wherein the population of therapeutic progenitor cells are generated from a population of HLA-defined hiPSCs.

11. The method of claim 10, wherein the population of HLA-defined hiPSCs comprise human CD34+ cord blood cells.

12. The method of claim 1, wherein the FF and XF culture of the hiPSCs in contact with the tankyrase/PARP inhibitor-continuing composition comprises culturing the hiPSCs on vitronectin-coated substrates.

13. The method of claim 1, wherein the FF and XF culture of the hiPSCs in contact with the tankyrase/PARP inhibitor-containing composition is performed for 1 to 10 passages.

14. The method of claim 13, wherein passages 1-3 of the FF and XF culture of the hiPSCs in contact with the tankyrase/PARP inhibitor-containing composition comprise selection of dome-shaped colonies for each subsequent passage.

15. The method of claim 1, wherein the FF and XF culture of the hiPSCs in contact with the tankyrase/PARP inhibitor-containing composition is performed for 4 to 10 passages.

16. The method of claim 1, further comprising cryopreserving the population of hiPSCs.

17. The method of claim 1, wherein the hiPSCs are derived from human somatic cells.

18. The method of claim 1, wherein the hiPSCs are derived from peripheral blood stem cells (PBSC) donors.

19. The method of claim 1, further comprising culturing the population of hiPSCs under conditions for promoting mesoderm determination.

20. The method of claim 1, further comprising culturing the population of hiPSCs under conditions for promoting hematovascular differentiation.
